SEQUENCE LISTING (75 sequences)

SEQ ID NO: 1 - DNA, 28 bp, synthetic construct
  misc_feature 21: note = RNA
  misc_feature 28: note = a blocking group
  source 1..28: mol_type = other DNA
  misc_feature join(1..20,22..28): note = DNA
gacggagccagagatttcctngtgatgv

SEQ ID NO: 2 - DNA, 20 bp, synthetic construct
  source 1..20: mol_type = other DNA
gacggagccagagatttcct

SEQ ID NO: 3 - DNA, 20 bp, synthetic construct
  source 1..20: mol_type = other DNA
aagctggtggcctttgagtt

SEQ ID NO: 4 - DNA, 28 bp, synthetic construct
  misc_feature 21: note = RNA
  misc_feature 28: note = a blocking group
  source 1..28: mol_type = other DNA
  misc_feature join(1..20,22..28): note = DNA
aagctggtggcctttgagttnccccacv

SEQ ID NO: 5 - DNA, 20 bp, synthetic construct
  source 1..20: mol_type = other DNA
aagctggtggcctttgagtt

SEQ ID NO: 6 - DNA, 30 bp, synthetic construct
  misc_feature 21: note = RNA
  misc_feature 30: note = a blocking group
  source 1..30: mol_type = other DNA
  misc_feature join(1..22,24..30): note = DNA
tgaacacatccccttctctcatnaaccaan

SEQ ID NO: 7 - DNA, 22 bp, synthetic construct
  source 1..22: mol_type = other DNA
tgaacacatccccttctctcat

SEQ ID NO: 8 - DNA, 22 bp, synthetic construct
  source 1..22: mol_type = other DNA
tgaacacatccccttctctcat

SEQ ID NO: 9 - DNA, 26 bp, synthetic construct
  misc_feature 19: note = RNA
  misc_feature 26: note = a blocking group
  source 1..26: mol_type = other DNA
  misc_feature join(1..18,20,26): note = DNA
ccagggctctgtgtgagcncccacav

SEQ ID NO: 10 - DNA, 16 bp, synthetic construct
  source 1..16: mol_type = other DNA
ccagggctctgtgtga

SEQ ID NO: 11 - DNA, 26 bp, synthetic construct
  misc_feature 19: note = RNA
  misc_feature 26: note = a blocking group
  source 1..26: mol_type = other DNA
  misc_feature join(1..18,20..26): note = DNA
gtgtgccggactgaccacnctgtggv

SEQ ID NO: 12 - DNA, 18 bp, synthetic construct
  source 1..18: mol_type = other DNA
gtgtgccggactgaccac

SEQ ID NO: 13 - DNA, 20 bp, synthetic construct
  source 1..20: mol_type = other DNA
gacacgcctggtaattgtcc

SEQ ID NO: 14 - DNA, 28 bp, synthetic construct
  misc_feature 21: note = RNA
  misc_feature 28: note = a blocking group
  source 1..28: mol_type = other DNA
  misc_feature join(1..20,22..28): note = DNA
gacacgcctggtaattgtccngcaggcv

SEQ ID NO: 15 - DNA, 20 bp, synthetic construct
  source 1..20: mol_type = other DNA
gacacgcctggtaattgtcc

SEQ ID NO: 16 - DNA, 28 bp, synthetic construct
  misc_feature 21: note = RNA
  misc_feature 28: note = a blocking group
  source 1..28: mol_type = other DNA
  misc_feature join(1..20,22..28): note = DNA
gcagcagagataatggaaacnatcaagv

SEQ ID NO: 17 - DNA, 20 bp, synthetic construct
  source 1..20: mol_type = other DNA
actcaacaatcacagcatcc

SEQ ID NO: 18 - DNA, 18 bp, synthetic construct
  source 1..18: mol_type = other DNA
aagtgtttgggcattttg

SEQ ID NO: 19 - DNA, 26 bp, synthetic construct
  misc_feature 19: note = RNA
  misc_feature 26: note = a blocking group
  source 1..26: mol_type = other DNA
  misc_feature join(1..18,20..26): note = DNA
aagtgtttgggcattttgnggggctv

SEQ ID NO: 20 - DNA, 18 bp, synthetic construct
  source 1..18: mol_type = other DNA
gcagcatgtaagggagac

SEQ ID NO: 21 - DNA, 27 bp, synthetic construct
  misc_feature 20: note = RNA
  misc_feature 27: note = a blocking group
  source 1..27: mol_type = other DNA
  misc_feature join(1..19,21..27): note = DNA
tggagaagattggggactancaaaccv

SEQ ID NO: 22 - DNA, 18 bp, synthetic construct
  source 1..18: mol_type = other DNA
catgtttgcctgggagat

SEQ ID NO: 23 - DNA, 19 bp, synthetic construct
  source 1..19: mol_type = other DNA
agctgtccatagggaggtt

SEQ ID NO: 24 - DNA, 27 bp, synthetic construct
  misc_feature 20: note = RNA
  misc_feature 27: note = a blocking group
  source 1..27: mol_type = other DNA
  misc_feature join(1..19,21..27): note = DNA
agctgtccatagggaggttngaatgtv

SEQ ID NO: 25 - DNA, 18 bp, synthetic construct
  source 1..18: mol_type = other DNA
aatggttgctgcactttg

SEQ ID NO: 26 - DNA, 27 bp, synthetic construct
  misc_feature 20: note = RNA
  misc_feature 27: note = a blocking group
  source 1..27: mol_type = other DNA
  misc_feature join(1..19,21..27): note = DNA
ctggttccaagtgatcgaangccaatv

SEQ ID NO: 27 - DNA, 20 bp, synthetic construct
  source 1..20: mol_type = other DNA
accactcttccaatgcctac

SEQ ID NO: 28 - DNA, 20 bp, synthetic construct
  source 1..20: mol_type = other DNA
aggaaagagttggtccttcc

SEQ ID NO: 29 - DNA, 28 bp, synthetic construct
  misc_feature 21: note = RNA
  misc_feature 28: note = a blocking group
  source 1..28: mol_type = other DNA
  misc_feature join(1..20,22..28): note = DNA
aggaaagagttggtccttccncgggtcv

SEQ ID NO: 30 - DNA, 19 bp, synthetic construct
  source 1..19: mol_type = other DNA
ctcatccaactgatcgagg

SEQ ID NO: 31 - DNA, 27 bp, synthetic construct
  misc_feature 20: note = RNA
  misc_feature 27: note = a blocking group
  source 1..27: mol_type = other DNA
  misc_feature join(1..19,21..27): note = DNA
ctctcagacctctgcaggangcctctv

SEQ ID NO: 32 - DNA, 18 bp, synthetic construct
  source 1..18: mol_type = other DNA
gctcttcttcctctgggc

SEQ ID NO: 33 - DNA, 19 bp, synthetic construct
  source 1..19: mol_type = other DNA
tagggtaggcgtatccgat

SEQ ID NO: 34 - DNA, 27 bp, synthetic construct
  misc_feature 20: note = RNA
  misc_feature 27: note = a blocking group
  source 1..27: mol_type = other DNA
  misc_feature join(1..19,21..27): note = DNA
tagggtaggcgtatccgatnggcctgv

SEQ ID NO: 35 - DNA, 18 bp, synthetic construct
  source 1..18: mol_type = other DNA
cagctctgcggttcaagt

SEQ ID NO: 36 - DNA, 26 bp, synthetic construct
  misc_feature 19: note = RNA
  misc_feature 26: note = a blocking group
  source 1..26: mol_type = other DNA
  misc_feature join(1..18,20..26): note = DNA
actgcaccatcaggaggtngggaccv

SEQ ID NO: 37 - DNA, 19 bp, synthetic construct
  source 1..19: mol_type = other DNA
cagtgtctgcaggtgtacc

SEQ ID NO: 38 - DNA, 18 bp, synthetic construct
  source 1..18: mol_type = other DNA
agctgtccggtcatagga

SEQ ID NO: 39 - DNA, 26 bp, synthetic construct
  misc_feature 19: note = RNA
  misc_feature 26: note = a blocking group
  source 1..26: mol_type = other DNA
  misc_feature join(1..18,20..26): note = DNA
agctgtccggtcataggangctgccv

SEQ ID NO: 40 - DNA, 18 bp, synthetic construct
  source 1..18: mol_type = other DNA
gctgctgaagagctggtt

SEQ ID NO: 41 - DNA, 27 bp, synthetic construct
  misc_feature 20: note = RNA
  misc_feature 27: note = a blocking group
  source 1..27: mol_type = other DNA
  misc_feature join(1..19,21..27): note = DNA
ccagctatgattcggatctnccctgtv

SEQ ID NO: 42 - DNA, 18 bp, synthetic construct
  source 1..18: mol_type = other DNA
aggaatcaggccaacatg

SEQ ID NO: 43 - DNA, 19 bp, synthetic construct
  source 1..19: mol_type = other DNA
aactgattttgttcacccg

SEQ ID NO: 44 - DNA, 27 bp, synthetic construct
  misc_feature 20: note = RNA
  misc_feature 27: note = a blocking group
  source 1..27: mol_type = other DNA
  misc_feature join(1..19,21..27): note = DNA
aactgattttgttcacccgngatcccv

SEQ ID NO: 45 - DNA, 19 bp, synthetic construct
  source 1..19: mol_type = other DNA
gacgatctgggacagactg

SEQ ID NO: 46 - DNA, 27 bp, synthetic construct
  misc_feature 20: note = RNA
  misc_feature 27: note = a blocking group
  source 1..27: mol_type = other DNA
  misc_feature join(1..19,21..27): note = DNA
actcggtcattctgctcaangcacgav

SEQ ID NO: 47 - DNA, 18 bp, synthetic construct
  source 1..18: mol_type = other DNA
atgctgacctcagaggga

SEQ ID NO: 48 - DNA, 20 bp, synthetic construct
  source 1..20: mol_type = other DNA
tccatctttgctaaaacctg

SEQ ID NO: 49 - DNA, 28 bp, synthetic construct
  misc_feature 21: note = RNA
  misc_feature 28: note = a blocking group
  source 1..28: mol_type = other DNA
  misc_feature join(1..20,22..28): note = DNA
tccatctttgctaaaacctgnatggatn

SEQ ID NO: 50 - DNA, 20 bp, synthetic construct
  source 1..20: mol_type = other DNA
cctgatggaggacaatttag

SEQ ID NO: 51 - DNA, 28 bp, synthetic construct
  misc_feature 21: note = RNA
  misc_feature 28: note = a blocking group
  source 1..28: mol_type = other DNA
  misc_feature join(1..20,22..28): note = DNA
cttacatgacgtcaatccctngtgaagv

SEQ ID NO: 52 - DNA, 19 bp, synthetic construct
  source 1..19: mol_type = other DNA
gtgaatgcttttcagggac

SEQ ID NO: 53 - DNA, 18 bp, synthetic construct
  source 1..18: mol_type = other DNA
acagcatccagctttgtc

SEQ ID NO: 54 - DNA, 26 bp, synthetic construct
  misc_feature 19: note = RNA
  misc_feature 26: note = a blocking group
  source 1..26: mol_type = other DNA
  misc_feature join(1..18,20..26): note = DNA
acagcatccagctttgtcnccattcv

SEQ ID NO: 55 - DNA, 20 bp, synthetic construct
  source 1..20: mol_type = other DNA
ccttggactgaagtaaagcc

SEQ ID NO: 56 - DNA, 26 bp, synthetic construct
  misc_feature 19: note = RNA
  misc_feature 26: note = a blocking group
  source 1..26: mol_type = other DNA
  misc_feature join(1..18,20..26): note = 19
atgttcgtcatgggtgtgnaaccagv

SEQ ID NO: 57 - DNA, 21 bp, synthetic construct
  source 1..21: mol_type = other DNA
agaagtatgacaacagcctca

SEQ ID NO: 58 - DNA, 19 bp, synthetic construct
  source 1..19: mol_type = other DNA
catgagtccttccacgata

SEQ ID NO: 59 - DNA, 27 bp, synthetic construct
  misc_feature 20: note = RNA
  misc_feature 27: note = a blocking group
  source 1..27: mol_type = other DNA
  misc_feature join(1..19,21..27): note = DNA
catgagtccttccacgatanccaaacv

SEQ ID NO: 60 - DNA, 19 bp, synthetic construct
  source 1..19: mol_type = other DNA
ccaaagttgtcatggatga

SEQ ID NO: 61 - DNA, 30 bp, synthetic construct
  misc_feature 23: note = RNA
  source 1..30: mol_type = other DNA
  misc_feature 30: note = a blocking group
  misc_feature join(1..22,24..30): note = DNA
agaagagctacgagctgcctgancggcccv

SEQ ID NO: 62 - DNA, 18 bp, synthetic construct
  source 1..18: mol_type = other DNA
catcaccattggcaatga

SEQ ID NO: 63 - DNA, 20 bp, synthetic construct
  source 1..20: mol_type = other DNA
tggagttgaaggtagtttcg

SEQ ID NO: 64 - DNA, 28 bp, synthetic construct
  misc_feature 21: note = RNA
  misc_feature 28: note = a blocking group
  source 1..28: mol_type = other DNA
  misc_feature join(1..20,22..28): note = DNA
  misc_feature 22: note = RNA (uracil)
tggagttgaaggtagtttcgntggattv

SEQ ID NO: 65 - DNA, 19 bp, synthetic construct
  source 1..19: mol_type = other DNA
aaggtagtttcgtggatgc

SEQ ID NO: 66 - DNA, 28 bp, synthetic construct
  misc_feature 21: note = RNA
  misc_feature 28: note = a blocking group
  source 1..28: mol_type = other DNA
  misc_feature join(1..20,22..28): note = DNA
tatgctcatgtggtgttgagngaaaggv

SEQ ID NO: 67 - DNA, 20 bp, synthetic construct
  source 1..20: mol_type = other DNA
agacattgacctcaccaaga

SEQ ID NO: 68 - DNA, 20 bp, synthetic construct
  source 1..20: mol_type = other DNA
aagaaccagtctgggatctt

SEQ ID NO: 69 - DNA, 28 bp, synthetic construct
  misc_feature 21: note = RNA
  misc_feature 28: note = a blocking group
  source 1..28: mol_type = other DNA
  misc_feature join(1..20,22..28): note = DNA
aagaaccagtctgggatcttngtactcv

SEQ ID NO: 70 - DNA, 18 bp, synthetic construct
  source 1..18: mol_type = other DNA
ttgtactggcgtggattc

SEQ ID NO: 71 - DNA, 26 bp, synthetic construct
  misc_feature 19: note = RNA
  misc_feature 26: note = a blocking group
  source 1..26: mol_type = other DNA
  misc_feature join(1..18,20..26): note = DNA
ctctgattaacgcccagcngttttgv

SEQ ID NO: 72 - DNA, 18 bp, synthetic construct
  source 1..18: mol_type = other DNA
ctctgattaacgcccagc

SEQ ID NO: 73 - DNA, 18 bp, synthetic construct
  source 1..18: mol_type = other DNA
gcggtcggtaaagttgtt

SEQ ID NO: 74 - DNA, 26 bp, synthetic construct
  misc_feature 19: note = RNA
  misc_feature 26: note = a blocking group
  source 1..26: mol_type = other DNA
  misc_feature join(1..18,20..26): note = DNA
gcggtcggtaaagttgttnccattav

SEQ ID NO: 75 - DNA, 18 bp, synthetic construct
  source 1..18: mol_type = other DNA
gcggtcggtaaagttgtt

(19) United States
(12) Patent Application Publication
(10) Pub. No.: US 2024/0344135 A1
(43) Pub. Date: Oct. 17, 2024

(54) A METHOD OF DETECTING A THYROID BIOMARKER

(71) Applicants: Agency for Science, Technology and Research, Singapore (SG); Nation University Hospital (Singapore) Pte Ltd, Singapore (SG)

(72) Inventors: Lian Chye Winston Koh, Singapore (SG); Peiling Yang, Singapore (SG)

(21) Appl. No.: 18/294,652

(22) PCT Filed: Aug. 5, 2022
(86) PCT No.: PCT/SG2022/050562

§ 371 Date: Feb. 2, 2024

(30) Foreign Application Priority Data
Aug. 5, 2021 (SG) 10202108592Y
Aug. 5, 2021 (SG) 10202108593R

(51) Int. Cl. C12Q 1/6886; C12Q 1/6806

(52) U.S. Cl. CPC C12Q 1/6886; C12Q 1/6806; C12Q 2600/112; C12Q 2600/118; C12Q 2600/158

(57) ABSTRACT

There is provided a method of detecting and/or determining the presence of one or more thyroid-specific nucleic acid, the method comprising annealing the one or more thyroid-specific nucleic acid in the presence of a control nucleic acid, and subjecting each of the one or more thyroid-specific nucleic acid to one or more amplification step in the presence of a mixture comprising a surfactant and an oligonucleotide primer and/or probe capable of hybridizing with the one or more thyroid-specific nucleic acid, wherein the oligonucleotide primer and/or probe comprises a cleavage site and a cleavable 3′ end. Also disclosed are methods of detecting and/or determining thyroid cancer recurrence and/or metastasis and thyroid-specific nucleic acid detection mixtures, and kits thereof.

### TECHNICAL FIELD

The present disclosure relates broadly to a method of detecting a thyroid-specific biomarker in a sample. In particular, the present disclosure relates to the detection, amplification, and quantification of a thyroid-specific nucleic acid.

### BACKGROUND

The current standard of care for management of thyroid cancer has been total thyroidectomy followed by post-operative adjuvant radioactive iodine (RAI) for majority of the patients. The decision for RAI and the I-131 dosage, ranging from 30 to 250 mCi per dose, is determined by the predicted tumour burden and risk of recurrence. In patients with advanced distant metastatic disease, higher doses of 100-250 mCi of RAI is usually recommended. There is a lack of precision for the estimation of adjuvant RAI treatment dosages due to the absence of a clinical tool that quantifies residual thyroid tissue volume reliably.

The current tumour marker, serum thyroglobulin that has a half-life of 65 hours, may take at least seven to 10 half-lives (four weeks) for complete thyroglobulin clearance in the absence of metastases. One study had shown that undetectable baseline thyroglobulin at one-month post-surgery was associated with the absence of structural disease on post-operative scans, whereas another known in the art study showed that undetectable thyroglobulin levels two to six months post-surgery was associated with radioactive iodine avid metastatic disease in 12% of the cases.

The role of post-surgical pre-ablative diagnostic radioiodine scan to estimate the remnant amount of thyroid tissue had been explored. However, there had been studies reporting that low doses of RAI given for these diagnoses were associated with higher risk of remnant ablation failure, hypothesized to be due to the effects of the RAI dose for diagnostic scan rendering lower RAI uptake for the subsequent treatment dose of RAI. As such, this is not routinely done in clinical practice.

Thyroid cancer surveillance for recurrence is performed using ultrasound scan of the neck, cross-sectional imaging in some patients, along with serum thyroglobulin (TG) to monitor disease burden in response to treatment. It detects recurrence in thyroid cancer with a sensitivity of 19-40% and specificity of 92-97%. The low sensitivity of thyroglobulin for detecting recurrence leaves room for the development of molecular tools.

Similarly, known in the art studies of papillary thyroid carcinoma cohort showed 24% of the patients ( 20/83) had anti-thyroglobulin antibodies (TG Ab). TG Ab interferes with the serum thyroglobulin immunometric assay (IMA) measurement causing TG underestimation, with the risk of missing detection of persistent or recurrent thyroid cancer. In this clinical setting, alternative methods of assessing serum TG using radioimmunoassay (RIA) or liquid chromatography/tandem mass spectrometry (LC-MS/MS) had been studied. These were reported to have no interference from TG Ab. However, the functional sensitivity of RIA (0.5-1.0 μg/L) and LC-MS/MS (1-2 μg/L) are lower than the IMA method (0.05-1.0 μg/L) and are not readily available at most institutions.

In this clinical scenario, TG Ab was used as a surrogate tumour marker for thyroid cancer. However, reduction of TG Ab levels post-treatment is usually delayed (half-life 10 weeks). The TG Ab level decreases in 75% of the patients following complete treatment, but only 50% of these patients have undetectable TG Ab after 4 years of follow-up. It is uncertain if this TG Ab persistence is due to continued TG production by persistent thyroid tissues not detected by imaging or a stigma of continued activity of plasma cells. Lastly, patients with poorly differentiated thyroid cancers lose the ability to produce thyroglobulin, making the measurement of thyroglobulin an unreliable reflection of tumour burden in these patients.

Therefore, there is a need to provide an alternative method to detect the presence of a thyroid-specific biomarkers.

### SUMMARY

In one aspect, the present invention provides a method of detecting and/or determining the presence of one or more thyroid-specific nucleic acid, the method comprising annealing the one or more thyroid-specific nucleic acid in the presence of a control nucleic acid, and subjecting each of the one or more thyroid-specific nucleic acid to one or more amplification step in the presence of a mixture comprising a surfactant and an oligonucleotide primer and/or probe capable of hybridizing with the one or more thyroid-specific nucleic acid, wherein the oligonucleotide primer and/or probe comprises a cleavage site and a cleavable 3′ end.

In some examples, wherein the amplification step of the nucleic acid is performed in the presence of three parts surfactant to one part amplification mixture.

In some examples, wherein method comprises two amplification steps.

In some examples, wherein the method further comprises a step of freeze and thawing the amplified mixture.

In some examples, wherein the method further comprises a step of freeze and thawing the amplified mixture between the one or more amplification steps.

In some examples, wherein the control nucleic acid is added to the sample at a constant amount to thereby normalizes of the amplification efficiency across a plurality of samples, optionally the control nucleic acid is added to the sample at about 10² to 10¹⁰ copies.

In some examples, wherein the amplification step includes interposing an annealing step between denaturation and priming.

In some examples, wherein the method further comprises a step of quantifying the amount of one or more thyroid-specific nucleic acid present in the sample and/or sequencing the target nucleic acid in the sample.

In some examples, wherein the one or more thyroid-specific nucleic acid is obtained from a biological sample.

In some examples, wherein the one or more thyroid-specific nucleic acid is obtained from plasma.

In some examples, wherein the one or more thyroid-specific nucleic acid is a cell free nucleic acid, optionally a circulating cell free nucleic acid, optionally a circulating cell free RNA.

In some examples, wherein the thyroid-specific nucleic acid are genes that are highly expressed and/or have four or more folds-change expression in the thyroid as compared to in other tissues.

In some examples, wherein the one or more thyroid-specific nucleic acid comprise thyroid peroxidase (TPO), thyroglobulin (TG), GFRA2 (glial cell line-derived neurotrophic factor family receptor alpha-2), IYD (iodotyrosine deiodinase), PDE8B (phosphodiesterase-8B), WDR86 (WD repeat domain 86), C16orf89 (Chromosome 16 Open Reading Frame 89), DGKI (Diacylglycerol kinase), DIO2 (Iodothyronine Deiodinase 2), TSHR (Thyroid Stimulating Hormone Receptor), and PAX8 (Paired box gene 8).

In some examples, wherein the one or more thyroid-specific nucleic acid comprises thyroid peroxidase (TPO), GFRA2 (glial cell line-derived neurotrophic factor family receptor alpha-2), IYD (iodotyrosine deiodinase), and thyroglobulin (TG).

In some examples, wherein the sample is obtained from a subject prior to and/or subsequent to a treatment, optionally the treatment is a surgery to remove thyroid and/or a radiation therapy.

In some examples, wherein the method comprises detecting and/or determining and/or quantifying the presence of the one or more thyroid-specific gene in a first sample and a second sample, wherein the first sample is taken at an earlier time point than the second sample, and wherein an increase in the presence of the one or more thyroid-specific gene in the second sample compared to the first sample indicates the subject to have residual thyroid tissue and/or tumour burden, or wherein an increase in the presence of the one or more thyroid-specific gene in the second sample compared to the first sample indicates the subject to have recurrence and/or of thyroid conditions.

In another aspect, the present invention provides a method of detecting and/or determining thyroid cancer recurrence and/or metastasis, the method comprising annealing the one or more thyroid-specific nucleic acid in the presence of a control nucleic acid, and subjecting each of the one or more thyroid-specific nucleic acid to one or more amplification step in the presence of a mixture comprising a surfactant and an oligonucleotide primer and/or probe capable of hybridizing with the one or more thyroid-specific nucleic acid, wherein the oligonucleotide primer and/or probe comprises a cleavage site and a cleavable 3′ end, wherein the method comprises detecting and/or determining and/or quantifying the presence of the one or more thyroid-specific gene in a first sample and a second sample, wherein the first sample is taken at an earlier time point than the second sample, and wherein an increase in the presence of the one or more thyroid-specific gene in the second sample compared to the first sample indicates the subject to have thyroid cancer recurrence and/or metastasis.

In some examples, wherein the one or more thyroid-specific gene comprises thyroid peroxidase (TPO), sodium-iodide symporter (NIS), thyroglobulin (TG), and thyroid stimulating hormone receptor (TSHR).

In some examples, wherein the sample is a plasma sample.

In yet another aspect, the present invention provides a thyroid-specific nucleic acid detection mixture comprising a first mixture comprising: a control nucleic acid, and a second mixture comprising: a surfactant, and an oligonucleotide primer and/or probe capable of hybridizing with a thyroid-specific nucleic acid, wherein the oligonucleotide primer and/or probe comprises a cleavage site and a cleavable 3′ end.

### Definitions

As used herein, the term “biological sample” refers to a sample obtained from a biological subject, including a sample of biological tissue or fluid origin obtained in vivo or in vitro. Hence, a “biological sample” may be a solid biological sample or a liquid biological sample. Examples of a “solid biological sample” may include biopsies, such as an organ biopsy, a tumour biopsy, stools, cell culture, food, plant extracts, and the like. Examples of a “fluid biological sample” or “liquid biological sample” include blood, serum, plasma, sputum, lavage fluid (for example peritoneal lavage), cerebrospinal fluid, urine, vaginal discharge, semen, sweat, tears, saliva, and the like. As used herein, the terms “blood”, “plasma”, and “serum” encompass fractions or processed portions thereof. Similarly, where a sample is taken from a biopsy, swab, smear, etc., the “sample encompass a processed fraction or portion derived from the biopsy, swab, smear, etc.

As used herein, the term “detecting” includes the step of determining the presence and/or absence of cfRNA. In some examples, the term “detecting” may further include the step of quantification of the cfRNA detected in the sample.

As used herein, the term “isolated” refers to a nucleic acid that is removed from its natural environment. An “isolated” nucleic acid is typically partially purified.

As used herein, the term “nucleic acid” refers to a nucleotide sequence that typically includes nucleotides comprising an A, G, C, T or U base. In some examples, nucleotide sequences may include other bases such as inosine, methylcytosine, hydroxymethylcytosine, methylinosine, methyladenosie and/or thiouridine, and the like. The term “nucleic acid” may include both single and/or double stranded deoxyribonucleic acid (DNA) and ribonucleic acid (RNA), including environmental DNA (eDNA), genomic DNA, bacterial DNA, viral DNA, cell-free DNA (cfDNA), complementary RNA (cRNA), messenger RNA (mRNA), transfer RNA (tRNA), microRNA (miRNA), cell free RNA (cfRNA), circulating tumour RNA (ctRNA), bacterial RNA, viral RNA, ribosomal RNA (rRNA) and the like.

As used in herein, the term “target nucleic acid” refers to nucleic acid whose presence is to be detected or measured or whose function, interactions or properties are to be studied. Therefore, a target nucleic acid includes essentially any nucleic acid for which a detectable probe (e.g., oligonucleotide probe) or assay exists, or can be produced or isolated by one skilled in the art. Target nucleic acid may include disease markers, viral DNA and/or RNA, bacterial DNA and/or RNA, tumour markers, and the like.

As used herein, the term “real time” refers to the actual time during which a process or event occurs and/or tracking of temporal changes and/or trajectories of cellular changes in samples drawn from different time points.

As used herein, the term “surfactant” refers to a composition that stabilizes water-in-oil droplets that is capable of or that can encapsulate nucleic acids (such as DNA, cDNA, cfDNA, RNA, cfRNA, and the like). In some examples, the surfactant may comprise a particular repeat unit comprising a perfluoropolyether and a polyalkylene oxide unit. In some examples, the surfactant may be one or more of fluorosurfactant, non-ionic surfactant, anionic surfactant, cationic surfactant, amphoteric surfactant, and the like. In some examples, the fluorosurfactant may be synthesized by coupling oligomeric perfluorinated polyethers (PFPE) with polyethyleneglycol (PEG).

The term “micro” as used herein is to be interpreted broadly to include dimensions from about 1 micron to about 1000 microns.

The term “nano” as used herein is to be interpreted broadly to include dimensions less than about 1000 nm.

The terms “coupled” or “connected” or “attached” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween. For example, the cleavage compound as described herein cleaves the oligonucleotide (e.g. primer, probe, and the like) within or adjacent to the cleavage domain. Thus, the term “adjacent” means that the cleavage compound cleaves the oligonucleotide at either the 5′-end or the 3′ end of the cleavage domain. In some examples of the present disclosure, the cleavage reactions yield a 5′-phosphate group and a 3′-OH group.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. As used herein, the term “substantially no” or “very low” refers to a sequence homology of less than at least 20%, or 19%, or 18%, or 17%, or 16%, or 15%, or 14%, or 13%, or 12%, or 11%, or 10%, or 9%, or 8%, or 7%, or 6%, or 5%, or 4%, or 3%, or 2%, or 1%, or 0.9%, or 0.8%, or 0.7%, or 0.6%, or 0.5%, or 0.4%, or 0.3%, or 0.2%, or 0.1%, or 0.01% sequence homology to the target nucleic acid (for example any human gene). In some examples, the term “substantially no” or “very low” sequence homology refers to the control gene having substantially different sequence to the target nucleic acid (for example any human gene). In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

The standard of care for thyroid cancer management is thyroidectomy and adjuvant radioactive iodine (RAI). There is a paucity of clinical tool that quantifies residual thyroid volume reliably for precise adjuvant RAI dosing. Serum thyroglobulin (TG), tumour marker for thyroid cancer, takes 4 weeks for complete clearance due to its long half-life, and might be undetectable in 12% of structural disease patients. It detects recurrence with a sensitivity of 19-40%, mainly attributed to issue of TG antibody interference with TG immunometric assay. The inventors of the present disclosure found that the quantity of thyroid-specific nucleic acid is indicative of amount of thyroid tissues, and that during thyroid surgery, nucleic acid levels decrease accordingly.

Exemplary, non-limiting embodiments of methods of amplifying and/or quantifying a thyroid biomarker are disclosed hereinafter. Also disclosed are methods of detecting and/or determining the presence and/or the amount of a thyroid biomarker.

In one aspect, there is provided a method of detecting and/or determining the presence of one or more thyroid-specific nucleic acid, the method comprising annealing the one or more thyroid-specific nucleic acid in the presence of a control nucleic acid, and subjecting each of the one or more thyroid-specific nucleic acid to one or more amplification step in the presence of a mixture comprising a surfactant and an oligonucleotide primer and/or probe capable of hybridizing with the one or more thyroid-specific nucleic acid, wherein the oligonucleotide primer and/or probe comprises a cleavage site and a cleavable 3′ end.

In some examples, the method comprises contacting the thyroid-specific nucleic acid with an annealing reagent comprising a primer of the thyroid-specific nucleic acid, a control nucleic acid, and an annealing mixture (including deoxyribonucleotide triphosphate (dNTP) mixes). In some examples, where the thyroid-specific nucleic acid is an RNA, the method comprises contacting the thyroid-specific nucleic acid with an annealing reagent comprising a reverse primer of the thyroid-specific nucleic acid, a control nucleic acid, and an annealing mixture (including deoxyribonucleotide triphosphate (dNTP) mixes).

In some examples, the annealing step precede the reverse transcription and amplification cycles.

In some examples, the amplification step of the nucleic acid is performed in the presence of three parts surfactant to one part amplification mixture.

In some examples, method comprises two amplification steps.

In some examples, the amplification step of the nucleic acid is performed in the presence of three parts surfactant to one part amplification mixture. In some examples, the amplification step is performed in an emulsion mixture. The emulsion mixture is made up of 1 to 10 parts of surfactant with 1 to 5 parts of amplification mixture, or 1 part of surfactant with 1 part of amplification mixture, or 2 parts of surfactant with 1 part of amplification mixture, or 3 parts of surfactant with 1 part of amplification mixture, or 4 parts of surfactant with 1 part of amplification mixture, or 5 parts of surfactant with 1 part of amplification mixture, or 6 parts of surfactant with 1 part of amplification mixture, or 7 parts of surfactant with 1 part of amplification mixture, or 8 parts of surfactant with 1 part of amplification mixture, or 9 parts of surfactant with 1 part of amplification mixture, or 10 parts of surfactant with 1 part of amplification mixture, or 3 parts of surfactant with 2 parts of amplification mixture, or 4 parts of surfactant with 2 parts of amplification mixture, or 5 parts of surfactant with 2 parts of amplification mixture, or 6 parts of surfactant with 2 parts of amplification mixture, or 7 parts of surfactant with 2 parts of amplification mixture, or 8 parts of surfactant with 2 parts of amplification mixture, or 9 parts of surfactant with 2 parts of amplification mixture, or 10 parts of surfactant with 2 parts of amplification mixture. In some examples, the emulsion is made up of 3 parts of surfactant with 1 part of amplification mixture. That is, the emulsion is made up of 3 parts of surfactant with 1 part of amplification mixture, when the amplification mixture is 10 μL, the surfactant is 30 μL, to thereby provide a total of 40 μL of emulsion mixture.

In some examples, the surfactant may be used at about 1% (w/w) to about 15% (w/w), or about 2, 3, 4, 5, 6, 7, 8, 9, 10, 11, 12, 13, 14, or 15% (w/w). In some examples, the surfactant may be 10% (w/w) of fluorosurfactant.

In some examples, method comprises two amplification steps.

In some examples, the method comprises 2, or 3, or 4, or 5 amplification steps. In some examples, the amplification cycle or step is repeated two times to five times. In some examples, the amplification step is repeated two times, or three times, or four times, or five times, or more. In some examples, the amplification step is repeated two times (i.e., two amplification cycles).

In some examples, the method may comprise adding an amplification (e.g., PCR) mixture to the thyroid-specific nucleic acid. In some examples, the mixture may comprise a DNA polymerase, an rhPCR mixture of the thyroid-specific nucleic acid, and an RNase (such as an RNase H2 enzyme).

In some examples, the method comprises generating an emulsion by adding 3 parts of surfactant to 1 part of PCR reaction mixture. In some examples, the method comprises mixing (such as vortexing) the emulsion generated until cloudy and uniform. In some examples, the amplification step may be a thermocycling reaction with enzyme activation, denaturation, annealing, and extension.

In some examples, the method comprises transferring the top fraction of the reaction mixture to a fresh tube. In some examples, the method further comprises topping up the fraction recovered with the same amount of polymerase (such as Taq polymerase) and RNase enzyme (such as RNase H2 enzyme) as used in the preceding PCR reaction.

In some examples, the method comprises a subsequent amplification step (e.g., a second or third or more thermocycling reactions) with enzyme activation, denaturation, hybridization, annealing and extension. In some examples, the method further comprises removing residual primers with an enzyme, followed by enzyme inactivation.

In some examples, the method further comprises a step of freeze and thawing the amplified mixture.

In some examples, the method further comprises a step of freeze and thawing the amplified mixture between the one or more amplification steps.

In some examples, the control nucleic acid is added to the sample at a constant amount to thereby normalizes of the amplification efficiency across a plurality of samples, optionally the control nucleic acid is added to the sample at about 10² to 10¹⁰ copies.

In some examples, the method further comprises a reverse transcription of the one or more thyroid-specific nucleic acid after annealing step.

In some examples, the method further comprises subjecting the thyroid-specific nucleic acid to reverse transcription. In some examples, the method further comprises a reverse transcription of the thyroid-specific nucleic acid after annealing step.

In some examples, the method comprises contacting the thyroid-specific nucleic acid with a reverse transcription agent comprising a reverse transcriptase.

In some examples, the method further comprises inactivation of the reverse transcriptase.

In some examples, the method further comprises a step of quantifying the amount of one or more thyroid-specific nucleic acid present in the sample and/or sequencing the target nucleic acid in the sample.

In some examples, the one or more thyroid-specific nucleic acid is obtained from a biological sample.

In some examples, the one or more thyroid-specific nucleic acid is obtained from plasma.

In some examples, the one or more thyroid-specific nucleic acid is a cell free nucleic acid, optionally a circulating cell free nucleic acid.

In some examples, the method further comprises the step of quantifying the one or more thyroid-specific nucleic acid.

In some examples, the method further comprises a step of quantifying the amount of target nucleic acid present in the sample and/or sequencing the target nucleic acid in the sample.

In some examples, the method of the present disclosure may be adaptable to include processing where amplified cDNA exhibits compatibility for downstream further processing. This is because the method of the present disclosure advantageously provides an adaptable end point where amplified cDNA exhibit compatibility for downstream quantification using methods known in the art. For example, the cDNA as amplified by the method as disclosed herein may be used in further steps of quantifying the amount of target nucleic acid by performing quantitative real-time PCR, next generation sequencing, UV absorbance with spectrophotometer, fluorescence dyes, agarose gel electrophoresis, microfluidic capillary electrophoresis, diphenylamine method, droplet digital PCR, and the like.

In some examples, the expression of the targeted nucleic acid may be monitored at different time point using qPCR.

In some examples, the thyroid-specific nucleic acid are genes that are highly expressed and/or have four or more folds-change expression in the thyroid as compared to in other tissues.

As used herein, the term “thyroid-specific nucleic acid” refers to nucleic acid that are highly expressed and/or have four or more folds-change expression in the thyroid as compared to in other tissues. In some examples, the expression is 2, 3, 4, 5, 6, 7, 8, 9, 10, or more folds-change. In some examples, the thyroid-specific target is biologically significant/relevant, and/or highly expressed (such as more than 2 to 10 times fold-change when compared to other tissues), and/or falls into the category of “tissue-enriched genes” in thyroid tissues. For example, highly expressed thyroid specific target when compared to other tissues may include, but is not limited to, 2 times fold-change, 3 times fold-change, 4 times fold-change, 5 times fold-change, 6 times fold-change, 7 times fold-change, 8 times fold-change, 9 times fold-change, or 10 times fold-change, and the like.

In some examples, the thyroid-specific target is biologically significant and/or highly expressed according to databases known in the art (such as Human Protein Atlas) and/or known in the art literature (such as published article). In some examples, the thyroid-specific target identified includes genes such as, but is not limited to, thyroid peroxidase (TPO), thyroglobulin (TG), GFRA2 (glial cell line-derived neurotrophic factor family receptor alpha-2), IYD (iodotyrosine deiodinase), PDE8B (phosphodiesterase-8B), WDR86 (WD repeat domain 86), C16orf89 (Chromosome 16 Open Reading Frame 89), DGKI (Diacylglycerol kinase), DIO2 (Iodothyronine Deiodinase 2), TSHR (Thyroid Stimulating Hormone Receptor), and PAX8 (Paired box gene 8), and the like.

In some examples, the one or more thyroid-specific nucleic acid comprise thyroid peroxidase (TPO), thyroglobulin (TG), GFRA2 (glial cell line-derived neurotrophic factor family receptor alpha-2), IYD (iodotyrosine deiodinase), PDE8B (phosphodiesterase-8B), WDR86 (WD repeat domain 86), C16orf89 (Chromosome 16 Open Reading Frame 89), DGKI (Diacylglycerol kinase), DIO2 (Iodothyronine Deiodinase 2), TSHR (Thyroid Stimulating Hormone Receptor), and PAX8 (Paired box gene 8).

In some examples, the method comprises detecting and/or determining the presence of two or more, three or more, four or more, five or more, six or more, seven or more, eight or more, nine or more, 10 or more, or 11 thyroid-specific nucleic acids.

In some examples, the one or more thyroid-specific nucleic acid comprises thyroid peroxidase (TPO), GFRA2 (glial cell line-derived neurotrophic factor family receptor alpha-2), IYD (iodotyrosine deiodinase), and thyroglobulin (TG).

In some examples, the method comprises detecting and/or determining the presence of two or more, three or more, or four thyroid-specific nucleic acid selected from the group consisting of thyroid peroxidase (TPO), GFRA2 (glial cell line-derived neurotrophic factor family receptor alpha-2), IYD (iodotyrosine deiodinase), and thyroglobulin (TG).

In some examples, the one or more thyroid-specific nucleic acid is detected using one or more of primers provided in the following table:

TABLE 1

| | | Reverse | | |
|---|---|---|---|---|
| Category | Targets | Primers | rh Primers | qPCR Primers |
| Thyroid | TG | | GACGGAGCCAGAGATTTCCTr | GACGGAGCC |
| | Forward | | GTGATG/3SpC3/ (SEQ ID NO: | AGAGATTTC |
| | | | 1) | CT (SEQ ID |
| | | | | NO: 2) |
| | TG | AAGCTGGT | AAGCTGGTGGCCTTTGAGTTr | AAGCTGGTG |
| | Reverse | GGCCTTTGA | CCCCAC/3SpC3/ (SEQ ID NO: | GCCTTTGAG |
| | | GTT (SEQ ID | 4) | TT (SEQ ID |
| | | NO: 3) | | NO: 5) |
| | IYD | | TGAACACATCCCCTTCTCTCA | TGAACACAT |
| | Forward | | TrAACCAA/3SpC3/ (SEQ ID | CCCCTTCTCT |
| | | | NO: 6) | CAT (SEQ ID |
| | | | | NO: 7) |
| | IYD | CCAGGGCT | CCAGGGCTCTGTGTGAGCrC | CCAGGGCTC |
| | Reverse | CTGTGTGA | CCACA/3SpC3/ (SEQ ID NO: 9) | TGTGTGA |
| | | (SEQ ID NO: | | (SEQ ID NO: |
| | | 8) | | 10) |
| | GFRA2 | | GTGTGCCGGACTGACCACrC | GTGTGCCGG |
| | Forward | | TGTGG/3SpC3/ (SEQ ID NO: | ACTGACCAC |
| | | | 11) | (SEQ ID NO: |
| | | | | 12) |
| | GFRA2 | GACACGCC | GACACGCCTGGTAATTGTCCr | GACACGCCT |
| | Reverse | TGGTAATTG | GCAGGC/3SpC3/ (SEQ ID NO: | GGTAATTGT |
| | | TCC (SEQ ID | 14) | CC (SEQ ID |
| | | NO: 13) | | NO: 15) |
| | TPO | | GCAGCAGAGATAATGGAAACr | ACTCAACAAT |
| | Forward | | ATCAAG/3SpC3/ (SEQ ID NO: | CACAGCATC |
| | | | 16) | C (SEQ ID NO: |
| | | | | 17) |
| | TPO | AAGTGTTTG | AAGTGTTTGGGCATTTTGrGG | GCAGCATGT |
| | Reverse | GGCATTTTG | GGCT/3SpC3/ (SEQ ID NO: 19) | AAGGGAGAC |
| | | (SEQ ID NO: | | (SEQ ID NO: |
| | | 18) | | 20) |
| | PAX8 | | TGGAGAAGATTGGGGACTArC | CATGTTTGC |
| | Forward | | AAACC/3SpC3/ (SEQ ID NO: | CTGGGAGAT |
| | | | 21) | (SEQ ID NO: |
| | | | | 22) |
| | PAX8 | AGCTGTCCA | AGCTGTCCATAGGGAGGTTrG | AATGGTTGC |
| | Reverse | TAGGGAGG | AATGT/3SpC3/ (SEQ ID NO: 24) | TGCACTTTG |
| | | TT (SEQ ID | | (SEQ ID NO: |
| | | NO: 23) | | 25) |
| | PDE8B | | CTGGTTCCAAGTGATCGAArG | ACCACTCTTC |
| | Forward | | CCAAT/3SpC3/ (SEQ ID NO: | CAATGCCTA |
| | | | 26) | C (SEQ ID NO: |
| | | | | 27) |
| | PDE8B | AGGAAAGA | AGGAAAGAGTTGGTCCTTCCr | CTCATCCAA |
| | Reverse | GTTGGTCCT | CGGGTC/3SpC3/ (SEQ ID NO: | CTGATCGAG |
| | | TCC (SEQ ID | 29) | G (SEQ ID NO: |
| | | NO: 28) | | 30) |
| | C16orf89 | | CTCTCAGACCTCTGCAGGArG | GCTCTTCTTC |
| | Forward | | CCTCT/3SpC3/ (SEQ ID NO: | CTCTGGGC |
| | | | 31) | (SEQ ID NO: |
| | | | | 32) |
| | C16orf89 | TAGGGTAG | TAGGGTAGGCGTATCCGATrG | CAGCTCTGC |
| | Reverse | GCGTATCC | GCCTG/3SpC3/ (SEQ ID NO: | GGTTCAAGT |
| | | GAT (SEQ ID | 34) | (SEQ ID NO: |
| | | NO: 33) | | 35) |
| | WDR86 | | ACTGCACCATCAGGAGGTrG | CAGTGTCTG |
| | Forward | | GGACC/3SpC3/ (SEQ ID NO: | CAGGTGTAC |
| | | | 36) | C (SEQ ID NO: |
| | | | | 37) |
| | WDR86 | AGCTGTCC | AGCTGTCCGGTCATAGGArG | GCTGCTGAA |
| | Reverse | GGTCATAG | CTGCC/3SpC3/ (SEQ ID NO: | GAGCTGGTT |
| | | GA (SEQ ID | 39) | (SEQ ID NO: |
| | | NO: 38) | | 40) |
| | DGKI | | CCAGCTATGATTCGGATCTrC | AGGAATCAG |
| | Forward | | CCTGT/3SpC3/ (SEQ ID NO: | GCCAACATG |
| | | | 41) | (SEQ ID NO: |
| | | | | 42) |
| | DGKI | AACTGATTT | AACTGATTTTGTTCACCCGrG | GACGATCTG |
| | Reverse | TGTTCACCC | ATCCC/3SpC3/ (SEQ ID NO: | GGACAGACT |
| | | G (SEQ ID | 44) | G (SEQ ID NO: |
| | | NO: 43) | | 45) |
| | DIO2 | | ACTCGGTCATTCTGCTCAArG | ATGCTGACC |
| | Forward | | CACGA/3SpC3/ (SEQ ID NO: | TCAGAGGGA |
| | | | 46) | (SEQ ID NO: |
| | | | | 47) |
| | DIO2 | TCCATCTTT | TCCATCTTTGCTAAAACCTGr | CCTGATGGA |
| | Reverse | GCTAAAACC | ATGGAT/3SpC3/ (SEQ ID NO: | GGACAATTTA |
| | | TG (SEQ ID | 49) | G (SEQ ID NO: |
| | | NO: 48) | | 50) |
| | TSHR | | CTTACATGACGTCAATCCCTr | GTGAATGCT |
| | Forward | | GTGAAG/3SpC3/ (SEQ ID NO: | TTTCAGGGA |
| | | | 51) | C (SEQ ID NO: |
| | | | | 52) |
| | TSHR | ACAGCATCC | ACAGCATCCAGCTTTGTCrCC | CCTTGGACT |
| | Reverse | AGCTTTGTC | ATTC/3SpC3/ (SEQ ID NO: 54) | GAAGTAAAG |
| | | (SEQ ID NO: | | CC (SEQ ID |
| | | 53) | | NO: 55) |
| Housekeeping | GAPDH | | ATGTTCGTCATGGGTGTGrAA | AGAAGTATG |
| | Forward | | CCAG/3SpC3/ (SEQ ID NO: 56) | ACAACAGCC |
| | | | | TCA (SEQ ID |
| | | | | NO: 57) |
| | GAPDH | CATGAGTCC | CATGAGTCCTTCCACGATArC | CCAAAGTTG |
| | Reverse | TTCCACGAT | CAAAC/3SpC3/ (SEQ ID NO: | TCATGGATG |
| | | A (SEQ ID | 59) | A (SEQ ID NO: |
| | | NO: 58) | | 60) |
| | ACTB | | AGAAGAGCTACGAGCTGCCT | CATCACCATT |
| | Forward | | GArCGGCCC/3SpC3/ (SEQ ID | GGCAATGA |
| | | | NO: 61) | (SEQ ID NO: |
| | | | | 62) |
| | ACTB | TGGAGTTGA | TGGAGTTGAAGGTAGTTTCGr | AAGGTAGTTT |
| | Reverse | AGGTAGTTT | UGGATT/3SpC3/ (SEQ ID NO: | CGTGGATGC |
| | | CG (SEQ ID | 64) | (SEQ ID NO: |
| | | NO: 63) | | 65) |
| | RPS18 | | TATGCTCATGTGGTGTTGAGr | AGACATTGA |
| | Forward | | GAAAGG/3SpC3/ (SEQ ID NO: | CCTCACCAA |
| | | | 66) | GA (SEQ ID |
| | | | | NO: 67) |
| | RPS18 | AAGAACCAG | AAGAACCAGTCTGGGATCTTr | TTGTACTGG |
| | Reverse | TCTGGGATC | GTACTC/3SpC3/ (SEQ ID NO: | CGTGGATTC |
| | | TT (SEQ ID | 69) | (SEQ ID NO: |
| | | NO: 68) | | 70) |
| Cont | LUC | | CTCTGATTAACGCCCAGCrGT | CTCTGATTAA |
| | Forward | | TTTG/3SpC3/ (SEQ ID NO: 71) | CGCCCAGC |
| | | | | (SEQ ID NO: |
| | | | | 72) |
| | LUC | GCGGTCGG | GCGGTCGGTAAAGTTGTTrCC | GCGGTCGGT |
| | Reverse | TAAAGTTGT | ATTA/3SpC3/ (SEQ ID NO: 74) | AAAGTTGTT |
| | | T (SEQ ID | | (SEQ ID NO: |
| | | NO: 73) | | 75) |

In some examples, the sample is obtained from a subject prior to and/or subsequent to a treatment, optionally the treatment is a surgery to remove thyroid and/or a radiation therapy.

In some examples, the subject may be a person suspected of or is having thyroid conditions. For example, the subject may be a person presenting with thyroid conditions and/or is a person undergoing thyroid surgery and/or a person having recurrent or persistent thyroid cancer. In some examples, the subject may be a person with a benign thyroid condition (e.g. benign thyroid nodules, hyperthyroidism from Graves' disease, and the like), or malignant thyroid nodules, and a person with recurrent/persistent thyroid cancer undergoing repeat thyroid surgery or radioactive iodine adjuvant therapy. In some examples, the subject may have or is suspected of having thyroid cancer. In some examples, the thyroid cancer may include, but is not limited to, a papillary thyroid cancer, a poorly differentiated thyroid cancer, a follicular thyroid cancer, and the like, and its combinations thereof.

In some examples, wherein the method comprises detecting and/or determining and/or quantifying the presence of the one or more thyroid-specific gene in a first sample and a second sample, wherein the first sample is taken at an earlier time point than the second sample, and wherein an increase in the presence of the one or more thyroid-specific gene in the second sample compared to the first sample indicates the subject to have residual thyroid tissue and/or tumour burden, or wherein an increase in the presence of the one or more thyroid-specific gene in the second sample compared to the first sample indicates the subject to have recurrence and/or of thyroid conditions.

Without wishing to be bound by theory, the detectable changes to the amount of nucleic acid (such as cfRNA) over time can advantageously be used as a real time indicator of the residual thyroid tissue volume. In some examples, the detectable changes can be observed over 6 to 8 hours. This is advantageous as compared to methods known in the art, which detects serum thyroglobulin that may take at least 4 weeks for complete thyroglobulin clearance in the absence of metastases.

In some examples, the method comprises collecting a sample. In some examples, the method comprises collecting a biological sample. For example, a biological sample may include a solid biological sample or a liquid biological sample. In some examples, the method comprises collecting a liquid biological sample. In some examples, the method comprises collecting blood (peripheral blood). In some examples, the method comprises collecting a sample in nucleic acid tubes (such as Streck cfRNA tubes) containing a mixture with a stabilizing reagent.

The inventors of the present disclosure had previously shown that cfRNA can change over 6 to 8 hours. As such, cfRNA could be a potential real-time indicator of the residual thyroid tissue volume as opposed to tumour marker, serum thyroglobulin that may take at least 4 weeks for complete thyroglobulin clearance in the absence of metastases. Therefore, in some examples, the samples may be obtained at real-time/different time points of the disease state. For example, the time points may include pre-treatment (e.g. pre-surgery or pre-RAI), peri-operative period, immediately after treatment, short term post-treatment, long-term post-treatment, antibody positive state, recurrent or persistent cancer (during surveillance visits and at the time of clinical recurrence), benign or metastatic cancer, and the like. For example, post-treatment may include, but is not limited to, 24 hours, 1 week, 2 weeks, 3 weeks, 1 month, 1.5 months, 2 months, 2.5 months, 3 months, 3.5 months, 4 months, 4.5 months, 5 months, 5.5 months, 6 months, 6.5 months, 7 months, 7.5 months, 8 months, 8.5 months, 9 months, 9.5 months, 10 months, 10.5 months, 11 months, 11.5 months, 12 months post-treatment, and the like. In some examples, real-time/different time points may include 24 hours post-treatment, one-month post-treatment, and six months post-treatment.

In some examples, the method may comprise detecting and/or determining and/or quantifying the presence of the one or more thyroid-specific gene at multiple time points. In some examples, the method may comprise detecting and/or determining and/or quantifying the presence of the one or more thyroid-specific gene at 24 hours, 1 week, 1 month, and 6 months post-treatment.

In some examples, the method comprises the extraction of a thyroid-specific nucleic acid from a sample. In some examples, residual nucleic acid (such as DNA in the cfRNA) was digested using an enzyme (such as RNase-free DNase I). In some examples, the extracted nucleic acid (such as cfRNA) was purified. In some examples, the method comprises yielding of at least 0-50 μl of thyroid-specific nucleic acid per sample. For example, the yield of thyroid-specific nucleic acid per sample may include, but is not limited to, 1 μl, 2 μl, 3 μl, 4 μl, 5 μl, 6 μl, 7 μl, 8 μl, 9 μl, 10 μl, 11 μl, 12 μl, 13 μl, 14 μl, 15 μl, 16 μl, 17 μl, 18 μl, 19 μl, 20 μl, 21 μl, 22 μl, 23 μl, 24 μl, 25 μl, 26 μl, 27 μl, 28 μl, 29 μl, 30 μl, 31 μl, 32 μl, 33 μl, 34 μl, 35 μl, 36 μl, 37 μl, 38 μl, 39 μl, 40 μl, 41 μl, 42 μl, 43 μl, 44 μl, 45 μl, 46 μl, 47 μl, 48 μl, 49 μl, or 50 μl, and the like.

In some examples, the method further comprises a step of confirming the presence of clinical recurrence by testing for one or more of serum thyroglobulin, thyroid uptake on radioiodine scan, physical examination of the neck, neck ultrasound, other imaging tests (for example, CT scan, MRI, and the like), and combinations thereof.

In some examples, the method may further comprise the step of treating the subject with the thyroid condition management/treatment. In some examples, the treatment may include, but is not limited to, a surgical removal of partial (i.e. hemi-thyroidectomy) or the entire thyroid gland (i.e. thyroidectomy), radiation, lymph node dissection, thyroid hormone therapy, alcohol ablation, targeted drug therapy, radiation therapy, chemotherapy, radiofrequency ablation, cryoablation, follow-up tests (e.g., physical examination of the neck, blood tests, ultrasound examination of the neck, other imaging tests e.g., CT, MRI), and the like. In some examples, the radiation may be subjecting the subject to a post-operative adjuvant radioactive iodine (RAI). In some examples, the radiation may include RAI and I-131 dosage ranging from about 30 to about 250 mCi per dose. In some examples, when the subject is determined to have advanced distant metastatic disease, the RAI and I-131 dosage may be about 100 to 250 mCi.

In some examples, the serum thyroglobulin may be measured using immunoassays known in the art. In some examples, the method further comprises measuring protein (such as serum TG) levels in a sample using a qualitative or quantitative assay. In some examples, the qualitative assay may include, but is not limited to, the Biuret test, the Burnt test, the Sakaguchi test, and the like. In some examples, the quantitative assay may include, but is not limited to, an immunometric assay, UV and visible spectroscopy, Bradford (BCA) assay, TG assay, radioimmunoassay, and the like.

In some examples, the method comprises a quantitative assay with a functional sensitivity between 0 μg/L to 5 μg/L. For example, the functional sensitivity may include, but is not limited to, 0.01 μg/L, 0.05 μg/L, 0.1 μg/L, 0.25 μg/L, 0.5 μg/L, 0.75 μg/L, 1 μg/L, 1.5 μg/L, 2 μg/L, 2.5 μg/L, 3 μg/L, 3.5 μg/L, 4 μg/L, 4.5 μg/L, 5 μg/L and the like. In some examples, the functional sensitivity is between 0.1 μg/L to 0.5 μg/L. In some examples, the method may comprise classifying a detectable level of thyroid-specific target (such as TG Ab) according to the functional sensitivity as TG Ab positive. In some examples, the method comprises the use of the reference values of a thyroid-specific target (such as TG Ab) to distinguish the presence or absence of thyroid disease/thyroid cancer/thyroid autoimmune disease.

In some examples, the method comprises a quantitative assay with analytical functional sensitivity between 0 to 50 IU/ml. For example, the analytical functional sensitivity may include, 0.1 IU/ml, 0.5 IU/ml, 1 IU/ml, 5 IU/ml, 10 IU/ml, 15 IU/ml, 20 IU/ml, 25 IU/ml, 30 IU/ml, 35 IU/ml, 40 IU/ml, 45 IU/ml, or 50 IU/ml. In some examples, the method comprises a quantitative assay (such as Immulite 2000 (Siemens), Roche) with analytical functional sensitivity of 20 IU/ml.

In some examples, the immunometric assay may comprise e411 (Roche), or E170 (Roche), with a functional sensitivity of 0.5 μg/L. In some examples, the TG assay may comprise Kryptor (Brahms) with a functional sensitivity of 0.15 μg/L, and/or e411 (Roche) with a functional sensitivity of 0.1 μg/L. In some examples, the radioimmunoassay may comprise Immunolite 2000 (Siemens) assay, and/or e411 (Roche).

Using circulating tumour DNA (ctDNA) to estimate residual thyroid cancer tissue can be challenging. In a study evaluating the performance of ctDNA using digital PCR, it was discovered that ctDNA was detected mainly in patients with solid tumours outside the brain (112 of 136; 82%). In contrast, less than 50% of patients with medulloblastomas or metastatic cancers of the kidney, prostate, and thyroid harboured detectable ctDNA.

Compared to ctDNA with only two copies per cell, multiple copies of tissue-specific RNA are present, providing a higher chance of detection. As such, cfRNA can potentially overcome the challenges of using small input amount of plasma and the need for higher sensitivity for detection of thyroid tissues. Unlike other solid organ malignancies where the primary organ is still in-situ after excision of tumour, thyroid cancer treatment involves removal of the entire thyroid gland as part of the treatment strategy. This would have led to the fall of thyroid-specific cell-free RNA (cfRNA) transcripts. Therefore any elevation in these circulating tissue-specific RNA levels could potentially indicate significant residual thyroid tissue or tumour burden since these transcripts reflect the presence of the cells originating from thyroid tissue.

Accordingly, in some examples, the method comprises assessing thyroid-specific nucleic acid (such as cDNA, DNA, RNA, cfRNA, and the like). For example, thyroid-specific nucleic acid may be a DNA and/or RNA.

In addition, the inventors of the present disclosure has found that cfRNA can advantageously address the issue of anti-thyroglobulin antibodies (present in 25% of thyroid cancer patients) that affects the reliability of thyroglobulin immunoassay. Accordingly, in some examples, thyroid-specific nucleic acid may be a cell free DNA and/or cell free RNA. In some examples, thyroid-specific nucleic acid may be a cell free RNA (cfRNA).

Furthermore, the inventors of the present disclosure identified that targeting circulating RNA of thyroid origin in thyroid cancer patients is a promising biomarker to monitor disease status. Utility of thyroid-specific mRNA transcripts such as thyroid peroxidase (TPO), sodium-iodide symporter (NIS), thyroglobulin (TG), and thyroid stimulating hormone receptor (TSHR) in predicting thyroid cancer recurrences and metastases had been assessed previously using whole blood. Therefore, in some examples, the thyroid-specific nucleic acid may be a circulating nucleic acid (such as circulating DNA and/or circulating RNA).

In some examples, the method further comprises tracking changes in thyroid-specific nucleic acid levels. For example, thyroid-specific nucleic acid level changes may include an increase or decrease in levels. In some examples, the method may comprise tracking for a decrease in thyroid-specific nucleic acid levels.

In some examples, the method comprises correlating thyroid-specific nucleic acid levels with methods of treating/diagnosing thyroid disease/thyroid cancer. In some examples, the method comprises correlating thyroid-specific nucleic acid levels with serum TG levels, radioactive scan (thyroid uptake), and neck ultrasound. In some examples, the method comprises classifying the treatment response. In some examples, the method comprises classifying the treatment response based on the American Thyroid Association (ATA) thyroid cancer management guidelines. In some examples, the treatment response may be classified as excellent, indeterminate, biochemical incomplete or structural incomplete.

In some examples, the method comprises classifying the treatment response based on the amounts of thyroid-specific target (such as after RAI ablation), and/or detection of the tumour with thyroid disease detection methods. In some examples, thyroid-specific nucleic acid may comprise a non-stimulated or stimulated protein (such as serum TG). In some examples, thyroid disease detection methods may comprise, but is not limited to, neck ultrasound scan, or cross-sectional or radioiodine imagining, and the like.

In some examples, the method comprises classifying the treatment response as excellent when non-stimulated protein (such as serum TG) is <0.2 μg/L or stimulated protein (such as serum TG) is <1.0 μg/L, and/or with no detectable thyroid-specific target (such as TG) antibodies, and/or no tumour/structural disease with neck ultrasound scan, and/or cross-sectional or radioiodine imaging.

In some examples, the method comprises classifying the treatment response as indeterminate when non-stimulated protein (such as serum TG) is between 0.2 μg/L and 1.0 μg/L or stimulated protein (such as serum TG) is ≥1-10 μg/L, and/or with stable or declining thyroid-specific target (such as TG) antibodies, and/or non-specific changes with neck ultrasound scan, and/or cross-sectional or radioiodine imaging.

In some examples, the method comprises classifying the treatment response as biochemical incomplete when non-stimulated protein (such as serum TG) is >1 μg/L or stimulated protein (such as serum TG) is >10 μg/L, and/or with increasing thyroid-specific target (such as TG) antibodies, and/or no tumour/structural disease with neck ultrasound scan, and/or cross-sectional or radioiodine imaging. In some examples, the method comprises classifying the treatment response as structural incomplete when patients have structural evidence of disease on imaging.

In some examples, the method further comprises a positive control comprising an RNA extracted directly from the thyroid.

In another aspect, there is provided a method of detecting and/or determining thyroid cancer recurrence and/or metastasis, the method comprising annealing the one or more thyroid-specific nucleic acid in the presence of a control nucleic acid, and subjecting each of the one or more thyroid-specific nucleic acid to one or more amplification step in the presence of a mixture comprising a surfactant and an oligonucleotide primer and/or probe capable of hybridizing with the one or more thyroid-specific nucleic acid, wherein the oligonucleotide primer and/or probe comprises a cleavage site and a cleavable 3′ end, wherein the method comprises detecting and/or determining and/or quantifying the presence of the one or more thyroid-specific gene in a first sample and a second sample, wherein the first sample is taken at an earlier time point than the second sample, and wherein an increase in the presence of the one or more thyroid-specific gene in the second sample compared to the first sample indicates the subject to have thyroid cancer recurrence and/or metastasis.

In some examples, the one or more thyroid-specific gene comprises thyroid peroxidase (TPO), sodium-iodide symporter (NIS), thyroglobulin (TG), and thyroid stimulating hormone receptor (TSHR).

TPO and TSHR mRNA extracted from whole blood showed significant correlation with disease status; they showed higher specificity (65-81%) but lower sensitivity (40-53%), while TG and NIS mRNA showed high sensitivity (60-73%) but low specificity (29-48%).

The inventors of the present disclosure also found that thyroid-specific cell free RNA (cfRNA) from the plasma fraction instead of whole blood can provide better sensitivity without the cellular background. Accordingly, in some examples, the sample is a plasma.

In another aspect, there is provided a thyroid-specific nucleic acid detection mixture and/or kit comprising a first mixture comprising: a control nucleic acid, and a second mixture comprising: a surfactant, and an oligonucleotide primer and/or probe capable of hybridizing with a thyroid-specific nucleic acid, wherein the oligonucleotide primer and/or probe comprises a cleavage site and a cleavable 3′ end. In some examples, the kit may comprise an instructions to perform the methods as disclosed herein.

Without wishing to be bound by theory, it is believed that the thyroid-specific cell free nucleic acid from plasma fraction, instead of whole blood, can provide better sensitivity without the cellular background.

In some examples, there is provided a method of amplification of a target nucleic acid, the method comprising annealing the target nucleic acid in the presence of a control nucleic acid, and subjecting the target nucleic acid to one or more amplification step in the presence of a mixture comprising a surfactant and an oligonucleotide primer and/or probe capable of hybridizing with the target nucleic acid, wherein the oligonucleotide primer and/or probe comprises a cleavage site and a cleavable 3′ end.

In some examples, the target nucleic acid may be a thyroid-specific nucleic acid.

In some examples, the amplification step of the nucleic acid is performed in the presence of three parts surfactant to one part amplification mixture. In some examples, the amplification step is performed in an emulsion mixture. The emulsion mixture is made up of 1 to 10 parts of surfactant with 1 to 5 parts of amplification mixture, or 1 part of surfactant with 1 part of amplification mixture, or 2 parts of surfactant with 1 part of amplification mixture, or 3 parts of surfactant with 1 part of amplification mixture, or 4 parts of surfactant with 1 part of amplification mixture, or 5 parts of surfactant with 1 part of amplification mixture, or 6 parts of surfactant with 1 part of amplification mixture, or 7 parts of surfactant with 1 part of amplification mixture, or 8 parts of surfactant with 1 part of amplification mixture, or 9 parts of surfactant with 1 part of amplification mixture, or 10 parts of surfactant with 1 part of amplification mixture, or 3 parts of surfactant with 2 parts of amplification mixture, or 4 parts of surfactant with 2 parts of amplification mixture, or 5 parts of surfactant with 2 parts of amplification mixture, or 6 parts of surfactant with 2 parts of amplification mixture, or 7 parts of surfactant with 2 parts of amplification mixture, or 8 parts of surfactant with 2 parts of amplification mixture, or 9 parts of surfactant with 2 parts of amplification mixture, or 10 parts of surfactant with 2 parts of amplification mixture. In some examples, the emulsion is made up of 3 parts of surfactant with 1 part of amplification mixture. That is, the emulsion is made up of 3 parts of surfactant with 1 part of amplification mixture, when the amplification mixture is 10 μL, the surfactant is 30 μL, to thereby provide a total of 40 μL of emulsion mixture.

In some examples, the surfactant may be used at about 1% (w/w) to about 15% (w/w), or about 2, 3, 4, 5, 6, 7, 8, 9, 10, 11, 12, 13, 14, or 15% (w/w). In some examples, the surfactant may be 10% (w/w) of fluorosurfactant.

In some examples, method comprises two amplification steps.

In some examples, the method comprises 2, or 3, or 4, or 5 amplification steps. In some examples, the amplification cycle or step is repeated two times to five times. In some examples, the amplification step is repeated two times, or three times, or four times, or five times, or more. In some examples, the amplification step is repeated two times (i.e. two amplification cycles).

In some examples, the method further comprises a step of freeze and thawing the amplified mixture.

In some examples, wherein the method further comprises a step of freeze and thawing the amplified mixture between the one or more amplification steps.

In some examples, the freeze and thawing step may be referred to as the emulsion breaking step. The inventors of the present disclosure found freeze thawing the emulsion PCR product advantageously provides for a robust, non-chemical based method of recovering the emulsion PCR product.

In some examples, the step of freezing comprises subjecting the mixture to a condition that freezes the mixture to a solid state. For example, the step of freezing subjects the mixture to a below freezing conditions. In some examples, the method comprises the step of freezing the mixture to 0° C. to −100° C., or to −50° C., or to −60° C., or to −70° C., or to −80° C. or to −90° C., or to −100° C. In some examples, the method comprises the step of freezing the mixture to −80° C.

In some examples, the method comprises freezing the reaction mixture for 0.5 hour to overnight. In some examples, the method comprises freezing the reaction mixture for 0.5 hour, or 1 hour, or 1.5 hour, or 2 hours, or 2.5 hours, or 3 hours, or 3.5 hours, or 4 hours, or 4.5 hours, or 5 hours, or 6 hours, or 7 hours, or 8 hours, or 9 hours, or overnight.

In some examples, the step of thawing comprises subjecting the mixture to a condition that allows the mixture to gain heat and change to a liquid state. For example, the step of thawing subjects the mixture to an above 0° C. conditions. In some examples, the step of thawing subjects the mixture to room temperature condition, for example from about 0° C. to about 40° C.

In some examples, the method may comprise adding an amplification (e.g. PCR) mixture to the target nucleic acid. In some examples, the mixture may comprise a DNA polymerase, a dNTP mixture, a cofactor (such as Magnesium Chloride), an rhPCR mixture of the target nucleic acid, and an RNase (such as an RNase H2 enzyme).

In some examples, the method comprises generating an emulsion by adding 3 parts of surfactant to 1 part of PCR reaction mixture. In some examples, the method comprises mixing (such as vortexing) the emulsion generated until uniform turbidity. In some examples, the amplification step may be a thermocycling reaction with enzyme activation, denaturation, annealing, and extension.

In some examples, the method comprises freezing the reaction mixture for 1 hour before thawing at room temperature. In some examples, the freeze thaw is performed between each amplification (thermocycling) step. In some examples, the method comprises transferring the top fraction of the reaction mixture to a fresh tube. In some examples, the method further comprises topping up the fraction recovered with the same amount of polymerase (such as Taq polymerase) and RNase enzyme (such as RNase H2 enzyme) as used in the preceding PCR reaction.

In some examples, the method comprises a subsequent amplification step (e.g. a second or third or more thermocycling reactions) with enzyme activation, denaturation, hybridization, annealing and extension. In some examples, the method further comprises removing residual primers with an enzyme, followed by enzyme inactivation.

In some examples, the oligonucleotide primer and/or probe capable of hybridizing with the target nucleic acid comprises a 5′ end of a functional primer, a cleavage site, one or more matching DNA bases, and one or more mismatch DNA base with one or more blocking group at the 3′ end.

In some examples, the primer may comprise about 10 to 40 bases, or 10 bases, 11 bases, 12 bases, 13 bases, 14 bases, 15 bases, 16 bases, 17 bases, 18 bases, 19 bases, 20 bases, 21 bases, 22 bases, 23 bases, 24 bases, 25 bases, 26 bases, 27 bases, 28 bases, 29 bases, 30 bases, 35 bases, or 40 bases.

In some examples, the functional primer may be about 16 to 24 bases, or about 16, 17, 18, 19, 20, 21, 22, 23, or 24 bases. In some examples, the reverse primers may comprise about 15 to 18 bases, or about 15, 16, 17, 18, 19, 20, 21, 22, 23, or 24 bases. In some examples, the qPCR primers may comprise about 15 to 20 bases, or 15, 17, 18, 19, 20, 21, 22, 23, or 24 bases

In some examples, the cleavage site is one or more RNA residue. In some examples, the cleavage site may comprise 2, 3, or 4 RNA residue. In some examples, the cleavage site is a single RNA residue or one RNA residue. In some examples, the cleavage site may be one or more of rU, rC, rG, or rA. In some examples, the cleavage site may be one of rC, rG, or rA.

In some examples, the cleavage site is one or more RNA residues.

In some examples, oligonucleotide primer and/or probe capable of hybridizing with the target nucleic acid comprises a 5′ end of a functional primer, a cleavage site consisting of one or more RNA residue, one or more matching DNA bases, and one or more mismatch DNA base with one or more blocking group at the 3′ end.

In some examples, the method comprises the step of cleaving the oligonucleotide primer and/or probe with an RNase enzyme.

In some examples, the cleavage site is cleaved by RNase H2 enzyme.

In some examples, the cleavage of the RNA residue releases the blocking group.

In some examples, the one or more matching DNA bases may comprise 1 DNA base, 2 DNA bases, 3 DNA bases, 4 DNA bases, 5 DNA bases, 6 DNA bases, 7 DNA bases, 8 DNA bases, 9 DNA bases, or 10 DNA bases. In some examples, the one or more matching DNA bases may be at the 3′ end of the cleavage site.

In some examples, the one or more mismatching DNA base may comprise 1 DNA base, 2 DNA bases, 3 DNA bases, 4 DNA bases, 5 DNA bases, 6 DNA bases, 7 DNA bases, 8 DNA bases, 9 DNA bases, or 10 DNA bases. In some examples, the one or more mismatching DNA base may be at the 3′ end of the matching DNA bases. In some examples, the primer may comprise one mismatching DNA base at the 3′ end of the primer.

In some examples, the primer and/or probe may comprise one or more blocking group. In some examples, the primer and/or probe may comprise 1, 2, 3, 4, 5, or more blocking groups. In some examples, the primer and/or probe may comprise 1 blocking group. In some examples, the primer and/or probe may comprise 2 blocking groups.

As disclosed herein, the blocking group may be a chemical moiety that is bound to the primer or other oligonucleotide such that an amplification reaction does not occur. For example, primer extension and/or DNA ligation does not occur. Once the blocking group is removed from the primer or other oligonucleotide, the oligonucleotide is capable of participating in the assay for which it was designed (e.g. PCR, ligation, sequencing, etc). Thus, the blocking group can be any chemical moiety that inhibits recognition by a polymerase or DNA ligase. The blocking group may be incorporated into the cleavage domain but is generally located on either the 5′- or 3′-side of the cleavage domain. In some examples, the blocking group is on the 3′ end of the oligonucleotide. The blocking group can be comprised of more than one chemical moiety. In the present invention the “blocking group” is typically removed after hybridization of the oligonucleotide to its target sequence. In some examples, the blocking group may be a C3 spacer (a phosphoramidite,

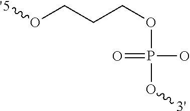

(for incorporation at 5′ end or internally), or

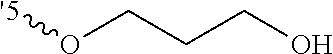

(for incorporation at 3′ end)), a hexanediol (a six carbon glycol spacer or

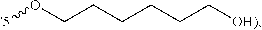

a 1′2′-dideoxiribose (dSpacer or

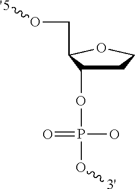

(for incorporation at 5′ end, internally, or 3′ end)), a PC Spacer (

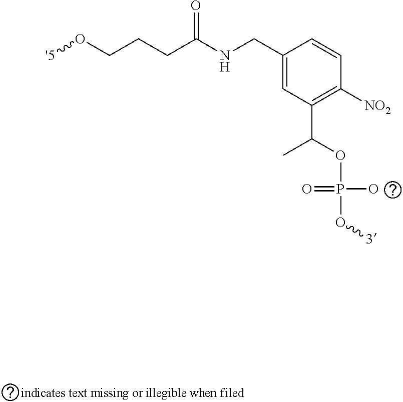

(for incorporation at the 5′ end or internally), a Spacer 9 (a thriethylene glycol spacer

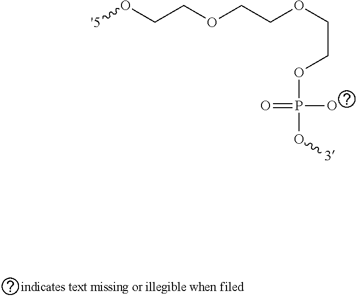

for incorporation at the 5′ end, internally, or 3′ end)), a Spacer 18 (18-atom hexa-ethyleneglycol spacer, or

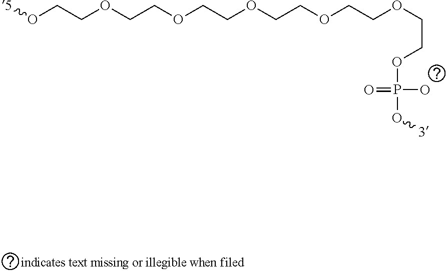

(for incorporation at the 5′ end, internally, or 3′ end)).

In some examples, the blocking group may be provided at the 3′ end of the primer. In some examples, the blocking group may be provided at the 3′ end of a mismatching DNA. In some examples, the blocking group may be provided within the one or more matching DNA bases and at the 5′ end of the mismatching DNA base. In some examples, where high fidelity of template amplification is desired, the blocking group is provided within the one or more matching DNA bases at the 5′ end of the mismatching DNA bases.

In some examples, the primer and/or probe may comprise formula (I):

Dₙ₁-Rₙ₂-Dₙ₃-Mₙ₄-Dₙ₃-x (I)
wherein
D is a DNA base that match target nucleic acid,
R is an RNA base that matches target,
M is a DNA base that is a mismatch to target nucleic acid,
X is a blocking group,
n1 is an integer representing the number of bases of between 1 to 20,
n2 is an integer representing the number of bases of between 1 to 5, and
n3 is an integer representing the number of bases of between 0 to 5.

In some examples, the primer and/or probe may comprise, in order from 5′ to 3′, a functional primer, a cleavage site, one or more matching DNA bases, one or more mismatch DNA base, and one or more blocking group.

In some examples, the primer and/or probe may comprise, in order from 5′ to 3′, a functional primer, a cleavage site, one or more matching DNA bases, one or more blocking group, one or more matching DNA bases, and one or more mismatching DNA.

In some examples, the primer comprising one or more RNA bases is an rhPCR primer (i.e. an RNase-dependent PCR primers), optionally the RNase-dependent PCR primers is an RNase H-dependent PCR primers. When the primer is an RNase H-dependent PCR primers, which is described in US 2015/225782 A1, the content of which is incorporated herein by reference.

The inventors of the present disclosure found that the combination of amplification of the target nucleic acid in surfactant (i.e. emulsion based PCR) with the oligonucleotide having cleavage site (i.e. rhPCR primers) advantageously increases the specificity of the amplification method.

In some examples, the present disclosure also includes the use of a probe. In some examples, the probe may be an oligonucleotide attached/conjugated to a detectable agent (such as a fluorophore and/or quencher). In some examples, the probe may be an oligonucleotide attached/conjugated to a detectable agent (such as a fluorescent label and/or quencher) and a groove binder. In some examples, the probe may comprise a nucleic acid binding reagent (such as SYBR® Green dye).

In some examples, the control nucleic acid is added to the sample at a constant amount to thereby normalizes of the amplification efficiency across a plurality of samples, optionally the control nucleic acid is added to the sample at about 10² to 10¹⁰ copies.

In some examples, the control nucleic acid (i.e. spike-in controls) does not compete or interfere with the amplification of the target nucleic acid. In some examples, the controls have low sequence homology to the target nucleic acid (for example it has low sequence homology to any human genes). In some examples, the control nucleic acid has a different sequence from the target nucleic acid. In some examples, the control nucleic acid is nucleic acid that cannot be found in the sample (i.e. exogenous from the sample) and/or is not a housekeeping gene. In some examples, the control nucleic acid is included in greater abundance than the target nucleic acid. The addition of a control nucleic acid allows for normalization of the technical amplification efficiency across samples. The control nucleic acid also advantageously normalizes for any unintended variation in the experiment.

In some examples, the control nucleic acid may be a DNA and/or RNA. In some examples, the control nucleic acid may be substantially no or very low sequence homology or substantially different from human gene. In some examples, the control nucleic acid may be luciferase. In some examples, the control nucleic acid may be luciferase RNA. As illustrated in the Experimental Section, the methods as disclosed herein may include the usage of luciferase RNA as a spiked-in for normalising PCR efficiency.

Without wishing to be bound by theory, it is believed that the method as disclosed herein may leverage on the “CoT effect” that increases the sensitivity of a method with minimal loss in linearity when used in quantitative methods. As used herein, the “CoT effect” refers to an amplification method where the presence of greater abundance of a particular nucleic acid results in a systemic bias against the more abundant of the two PCR products (one being an abundant nucleic acid (may be an internal control or an endogenous nucleic acid present in abundance in the sample) and the other being the target nucleic acid). The slowdown in amplification of abundant products allows the target nucleic acid/target of interest (which may be present in less quantity) to become more visible in the fingerprint. It is believed that the increase visibility of the target nucleic acid/target of interest (which may be present in lesser quantities) allow the target of interest (such as rarer cDNAs) to be sampled more efficiently. In another word, CoT PCR enable selective amplification of low concentration DNA resulting in increase of sensitivity for downstream applications.

In some examples, the methods as disclosed herein may comprise CoT PCR. In some examples, the amplification step in the method as described herein includes interposing an annealing step between denaturation and priming. Without wishing to be bound by theory, since the amount of re-annealing depends on the product of the initial concentration and time, CoT, the more abundant a sequence the greater will be the extent of its conversion to the double-stranded form. These hybrids will then fail to be copied in the polymerisation step of the cycle. Thus, as each sequence reaches the threshold concentration at which considerable re-annealing occurs it ceases to be exponentially amplified, and in this way, all sequences will eventually have the same concentration. In some examples, the CoT PCR maybe as described by Brenner S. and Jones DSC, 1972 (Wellcome collection, which can be accessed here: https://wellcomecollection.org/works/h39jksrt/items, the content of which is incorporated herein).

In some examples, the method comprises a subsequent amplification step (e.g., a second or third or more thermocycling reactions) with enzyme activation, denaturation, hybridization, annealing and extension.

Advantageously, CoT PCR enrichment may preferentially amplify rare amplicons over abundant ones by taking advantage of the CoT effect.

In a comparative example, a combination of emulsion rhPCR and CoT is shown to decrease the number of PCR cycles required for detection of a target polynucleotide as compared to emulsion rhPCR without CoT. Thus, a combination of emulsion rhPCR and CoT may advantageously increase a sensitivity of embodiments of the method in determining, detecting or quantifying a pancreas-associated polynucleotide. This allows embodiments of the method to detect or quantify low levels of pancreas-associated polynucleotide in a subject, which may not be possible otherwise.

Without wishing to be bound by theory, it is believed that the Cot effect is described by a faster rate of re-association of higher abundance single stranded DNA (ssDNA) to double-stranded DNA (dsDNA). Using this phenomenon, one way to enrich for rare target is to allow the higher abundant ssDNA to reassociate to dsDNA and then follow up with a method to remove the dsDNA to achieve removal of abundant dsDNA. In the present disclosure however, embodiments of the method do not involve removing the abundant dsDNA that was amplified (e.g., by rhPCR). Instead, the less abundant DNA in the reaction is allowed to have a higher probability of amplified. To achieve this, the CoT phenomenon may be implemented during amplification (e.g., during rhPCR) by adjusting/controlling the thermal cycling profile of the amplification process (e.g., rhPCR). The PCR reaction mix (e.g., the rhPCR reaction mix) may be held at the melting temperature of the dsDNA amplicons during the denaturing step of the PCR (e.g., rhPCR). Without wishing to be bound by theory, it is believed that, when the PCR reaction is being held at the melting temperature of the dsDNA amplicon, the CoT effect kicks in: the abundant dsDNA preferentially remains double stranded, and only dsDNA amplicons at low concentration will dissociate. These dissociated ssDNA are the only ones accessible to primers in the subsequent annealing and extension step which completes the PCR. By repeating this every cycle (i.e., holding the PCR reaction at the melting temperature of the dsDNA amplicon at the denaturing step), the initial low abundance amplicons will amplify up to a point where it becomes suitably abundant and joins other high abundance amplicons and be inhibited from disassociation, which allows for other remaining low abundance amplicons to be amplified.

In some examples, the control nucleic acid is a nucleic acid that may be added to the method as disclosed herein in a fixed amount (or a constant amount in all samples). In some examples, the control nucleic acid is provided at a concentration that is higher than the predicted concentration of the target of interest (or target nucleic acid).

In some examples, the amount of control nucleic acid added to the sample is about 10² to 10¹⁰ copies. In some examples, the amount of control nucleic acid maybe about 100 copies, 10³ copies, 10⁴ copies, 10⁵ copies, 10⁶ copies, 10⁷ copies, 10⁸ copies, 10⁹ copies, or 10¹⁰ copies. In some examples, the amount of control nucleic acid may be about 100 to 10⁹ copies, or about 100 to 10⁸ copies, or about 100 to 10⁷ copies, or about 100 to 10⁶ copies, or about 100 to 10⁵ copies. In some examples, the amount of control nucleic acid is about 10⁵ copies.

In some examples, the method may further comprise the detection of a second control nucleic acid that is present endogenously in the sample. For example, the second control nucleic acid may be a housekeeping gene. In some examples, the second control nucleic acid may include, but is not limited to, actin beta (ACTB), glyceraldehyde 3-phosphate dehydrogenase (GAPDH), ribosomal protein S18 (RPS18), ubiquitin C (UBC), beta-2 microglobulin (B2M), glucuronidase beta (GUSB), hypoxanthine-guanine phosphoribosyltransferase (HPRT), phosphoglycerate kinase 1 (PGK1), peptidylprolyl isomerase A (PPIA), TATA box binding protein (TBP), transferrin receptor (TFRC), tyrosine 3-monoxygenase/tryptophan 5-monooxygenase activation protein zeta (YWHAZ), tubulin, heat shock protein 90 (HSP90), hypoxanthine guanine phosphoribosyl transferase (HPRT), succinate dehydrogenase complex, subunit A (SDHA), mitochondrially encoded 12S ribosomal RNA (mtRNR1), mitochondrially encoded 16S RNA (mtRNR2), and the like. The detection of the second control nucleic acid allows for the normalization of extraction efficiency across samples.

In some examples, the method further comprises analysing data by normalizing raw values (such as Ct value) to the levels of control nucleic acids (such as housekeeping gene, spiked in luciferase RNA).

In some examples, the method comprises contacting the target nucleic acid with an annealing reagent comprising a primer of the target nucleic acid, a control nucleic acid, and an annealing mixture (including deoxyribonucleotide triphosphate (dNTP) mixes). In some examples, where the nucleic acid is an RNA, the method comprises contacting the target nucleic acid with an annealing reagent comprising a reverse primer of the target nucleic acid, a control nucleic acid, and an annealing mixture (including deoxyribonucleotide triphosphate (dNTP) mixes).

In some examples, the annealing step precede the reverse transcription and amplification cycles.

In some examples, the method further comprises subjecting the target nucleic acid to reverse transcription. In some examples, the method further comprises a reverse transcription of the target nucleic acid after annealing step.

In some examples, the method comprises contacting the target nucleic acid with a reverse transcription agent comprising a reverse transcriptase, and a reverse transcriptase mixture (including DTT).

In some examples, the method further comprises inactivation of the reverse transcriptase.

In some examples, the method further comprises a step of quantifying the amount of target nucleic acid present in the sample and/or sequencing the target nucleic acid in the sample.

In some examples, the method of the present disclosure may be adaptable to include processing where amplified cDNA exhibits compatibility for downstream further processing. This is because the method of the present disclosure advantageously provides an adaptable end point where amplified cDNA exhibit compatibility for downstream quantification using methods known in the art. For example, the cDNA as amplified by the method as disclosed herein may be used in further steps of quantifying the amount of target nucleic acid by performing quantitative real-time PCR, next generation sequencing, UV absorbance with spectrophotometer, fluorescence dyes, agarose gel electrophoresis, microfluidic capillary electrophoresis, diphenylamine method, droplet digital PCR, and the like.

Diverse RNA transcripts are widely detected to be circulating within the human plasma. The notion of using these circulating cell free RNA (cfRNA) as potential biomarkers has recently emerged from comprehensive assessments using high throughout sequencing technologies that led to the identification of tissue-specific cfRNA changes that correlate to pathological conditions such as cancer and metabolic diseases. Follow up work by the inventors have also shown that tissue specific cfRNA provides a non-invasive window for studying hard to reach tissues under different biological conditions.

Quantification of tissue-specific cfRNA is expected to vary based on the tissue of origin and the biological state of the cells when releasing cfRNA via apoptosis. However, the lack of effective and sensitive molecular tools to amplify and quantitate RNA biases against low abundance tissue specific cfRNA of interest. This in turn limits the widespread use of cfRNA as biomarkers. To address this, the present disclosure discloses a molecular protocol that overcome this by combining emulsion-based PCR together with specifically designed rhprimers that pre-amplifies tissue specific cfRNA for downstream quantitation with qPCR, or next generation sequencing.

Therefore, in some examples, the nucleic acid is a cell free nucleic acid, optionally a circulating cell free nucleic acid. In some examples, the cell free nucleic acid is a cell free DNA and/or a cell free RNA. In some examples, the nucleic acid is a circulating cell free RNA. In some examples, the cell free nucleic acid is an isolated cell free nucleic acid.

In some examples, when the nucleic acid is an RNA, the method comprises annealing the target nucleic acid is in the presence of a reverse primer of the target nucleic acid and the control nucleic acid, subjecting the annealed sample to reverse transcription, and subjecting the target nucleic acid to one or more amplification step in the presence of a mixture comprising a surfactant and a primer comprising one or more RNA base and a cleavable 3′ end.

In some examples, the reverse primers of the target nucleic acid may comprise about 10 to 40 bases, or 10 bases, 11 bases, 12 bases, 13 bases, 14 bases, 15 bases, 16 bases, 17 bases, 18 bases, 19 bases, 20 bases, 21 bases, 22 bases, 23 bases, 24 bases, 25 bases, 26 bases, 27 bases, 28 bases, 29 bases, 30 bases, 35 bases, or 40 bases. In some examples, the reverse primers of the target nucleic acid may be about 15-18 bases, or about 16 bases.

In some examples, the method further comprises the extraction of the nucleic acid from a sample.

In some examples, the sample may include any items that may contain nucleic acid of interest. For example, the items may be a surface of an equipment, a laboratory bench, a public surface (such as, but not limited to, surface on an elevator/lift/doorknobs/toilet, surface on a public transport, surface of airport areas, surface of school areas, surface of shopping mall or supermarket areas, surface of restaurants/hawkers/cafes, and the like), frequently touched surfaces adjacent to patients in hospitals/clinics (such as, but not limited to, areas adjacent to or at the hospital bed, hospital/clinic waiting areas, quarantine rooms and the like).

In some examples, the sample may be a biological sample. In some examples, the nucleic acid is obtained from a biological sample.

In some examples, the samples may be obtained at different time points of the disease state. For examples, the disease state may include pre-surgery, peri-operative period, immediately after surgery, short term post-surgery, long-term post-surgery, antibody positive state, recurrent or persistent cancer, and the like.

The presence or absence of a target nucleic acid can be measured quantitatively or qualitatively. Target nucleic acid can come in a variety of different forms including, for example, simple or complex mixtures, or in substantially purified forms. For example, a target nucleic acid can be a part of a sample that contains other components or can be the sole or major component of the sample. Therefore, a target nucleic acid can be a component of the circulatory system (such as blood, serum, plasma, or combinations thereof), a whole cell or tissue, a cell or tissue extract, a fractionated lysate thereof or a substantially purified molecule. In some examples, the target nucleic acid is a region of interest in a cell free DNA and/or RNA. In some examples, the target nucleic acid is a region of interest in a cell free RNA. In some examples, the target nucleic acid is a region of interest in a circulating cell free RNA.

In some examples, the method detects the presence and/or absence of any one of the following interest, such as, but not limited to, a pathogen, a disease, a cancer, a genetic defect, and the like. For example, pathogens may be a bacterial pathogen, a viral pathogen, a fungal pathogen, or a parasite.

Examples of a bacterial pathogen may include, but is not limited to, Escherichia coli, Mycobacteria spp, Salmonella spp, Staphylococcus spp, Clostridium difficile, Listeria monocytogenes, Group B streptococci, vancomycin-resistant enterococci (VRE), and the like.

Examples of a viral pathogen may include, but is not limited to, Human papillomavirus, Rhinovirus, Human cytomegalovirus in HIV-1 positive patient, Hepatitis virus, Coronavirus (CoV), severe acute respiratory syndrome (SARS), monkey pox virus and the like.

Examples of a fungal pathogen may include, but is not limited to, Botrytis cinerea, Pseudomonas syringae, Fusarium oxysporum and the like.

Examples of a parasite may include, but is not limited to, Leishmania parasites, Giardia, Cryptosporidium, Entamoeba and the like.

In some examples, the disease may be a metabolic disorder, such as, but is not limited to, hypothyroidism, hyperthyroidism, diabetes, mitochondrial disorders, phenylketonuria (PKU), and the like.

In some examples, the cancer may include, but is not limited to, thyroid cancer, pancreatic cancer, breast cancer, colon cancer, lung cancer, liver cancer, skin cancer, and the like.

In some examples, the genetic defects may include, but is not limited to, a prenatal genetic defect, Cystic fibrosis, and the like. In some examples, the prenatal genetic defect may include, but is not limited to, Down syndrome (Trisomy 21), Turner Syndrome, Edwards' syndrome, and the like.

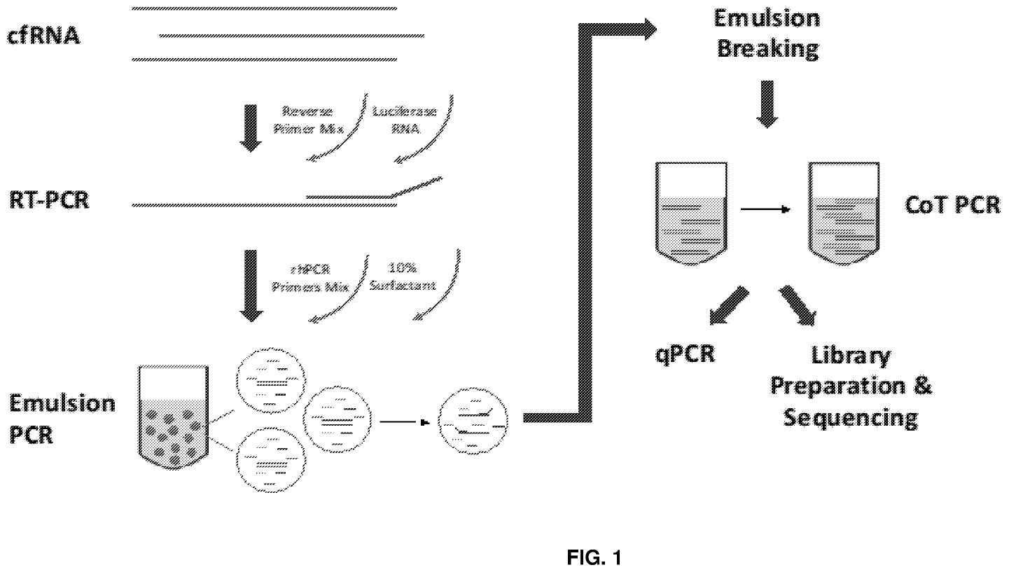

The present invention can advantageously be performed as a “one-pot amplification” process. The one-pot amplification made possible by the use of the emulsion PCR and the spiking of the sample with a control nucleic acid. As illustrated in FIG. 1, the methods as disclosed herein can be performed in a single closed tube from annealing step to reverse transcription and amplification step.

In some examples, the method is a real time amplification method.

In some examples, the method as disclosed herein may be performed on samples drawn at multiple time points. In some examples, the samples may be drawn/obtained from the subject at one or more, 2 or more, 3 or more, 4 or more, 5 or more, 6 or more, 7 or more, 8 or more, 9 or more, or 10 or more time points. In some examples, the samples may be drawn/obtained from the subject at 1 to 10 time points, or at least at 1 time point, or at least at 2 time points, or at least at 3 time points, or at least at 4 time points, or at least at 5 time points, or at least at 6 time points, or at least at 7 time points, or at least at 8 time points, or at least at 9 time points, or at least at 10 time points. In some examples, the samples may be drawn/obtained from the subject at at least 5 time points. For example, the 5 time points may include pre-surgery, short-term post-surgery, long-term post-surgery, and the like.

In another aspect, there is provided a nucleic acid amplification mixture comprising a first mixture comprising: a control nucleic acid, and a second mixture comprising: a surfactant, and an oligonucleotide primer and/or probe capable of hybridizing with a target nucleic acid, wherein the oligonucleotide primer and/or probe comprises a cleavage site and a cleavable 3′ end.

In some examples, the second mixture further comprises amplification reagents.

In some examples, wherein the amplification agent comprises detectable primers and/or probes.

In some examples, there is provided a kit comprising the reagents and/or mixtures used in the methods as disclosed herein.

In some examples, the target nucleic acid may be present in the sample in minute amount or in low quantity. In some examples, the target nucleic acid may not be present in abundance.

In some examples, the amount of sample nucleic acid may be about 1 μL to about 100 μL. In some examples, the amount of cfRNA in the sample may be about 1 μL to 90 μL, or about 5 μL to about 80 μL, or about 10 μL to about 50 μL. In some examples, the amount of cfRNA in the sample may be no more than 50 μL, no more than 40 μL, no more than 30 μL, no more than 20 μL, no more than 19 μL, no more than 18 μL, no more than 17 μL, no more than 16 μL, no more than 15 μL, no more than 14 μL, no more than 13 μL, no more than 12 μL, no more than 11 μL, no more than 10 μL, and the like.

In some examples, the amount of sample nucleic acid may be about 500 picogram (pg) to about 1000 μg. In some examples, the amount of sample nucleic acid may be about 500 pg, may be about 550 pg, may be about 600 pg, may be about 650 pg, may be about 700 pg, may be about 750 pg, may be about 800 pg, may be about 850 pg, may be about 900 pg, may be about 950 pg, may be about 1000 pg, may be about 1050 pg, may be about 1100 pg, may be about 1150 pg, may be about 1200 pg, may be about 1300 pg, may be about 1400 pg, may be about 1500 pg, may be about 2000 pg, may be about 3000 pg, may be about 4000 pg, may be about 5000 pg, may be about 6000 pg, may be about 7000 pg, may be about 8000 pg, may be about 9000 pg, may be about 10,000 pg, may be about 15,000 pg, may be about 20,000 pg, may be about 25,000 pg, may be about 30,000 pg, may be about 35,000 pg, may be about 40,000 pg, may be about 50,000 pg, may be about 100,000 pg, may be about 0.01 μg, may be about 0.02 μg, may be about 0.03 μg, may be about 0.04 μg, may be about 0.05 μg, may be about 0.06 μg, may be about 0.07 μg, may be about 0.08 μg, may be about 0.09 μg, may be about 0.1 μg, may be about 0.2 μg, may be about 0.3 μg, may be about 0.4 μg, may be about 0.5 μg, may be about 0.6 μg, may be about 0.7 μg, may be about 0.8 μg, may be about 0.9 μg, or may be about 1 μg. In some examples, the amount of sample nucleic acid may be no more than 2 μg, 3 μg, 4 μg, 5 μg, 6 μg, 7 μg, 8 μg, 9 μg, 10 μg, 11 μg, 12 μg, 13 μg, 14 μg, 15 μg, 20 μg, 30 μg, 40 μg, 50 μg, 100 μg, 150 μg, 200 μg, 300 μg, 400 μg, or 500 μg, or 2 to 1000 μg.

In another aspect, there is provided a method of detecting and/or determining the presence and/or the amount of a target nucleic acid comprising annealing the target nucleic acid in the presence of a control nucleic acid, and subjecting the target nucleic acid to one or more amplification step in the presence of a mixture comprising a surfactant and an oligonucleotide capable of hybridizing to the target nucleic acid comprising one or more RNA base and a cleavable 3′ end.

Also disclosed are methods of quantifying tissue/organ health in disease states. For example, the methods as disclosed herein may be applied to metabolic disorders and cancer surveillance.

As such, the present disclosure provides sensitive and multiplex methods for targeted amplification and/or quantification of low amounts of naturally occurring tissue specific RNA extracted from plasma.

In some examples, the method may comprise: (a) providing a reaction mixture comprising (i) rhPCR primers (e.g. an oligonucleotide primer having a cleavage domain positioned 5′ of a blocking group and 3′ of a position of variant, the blocking group linked at or near the end of the 3′-end of the oligonucleotide primer wherein the blocking group prevents primer extension and/or inhibits the oligonucleotide primer from serving as a template for DNA synthesis), (ii) a control RNA, (b) subjecting the reaction mixture to reverse transcription conditions, (c) contacting the resulting mixture from step (b) with a surfactant and an amplification mixture, (d) subjecting the mixture from step (c) to amplification conditions sufficient to result in the amplification of the nucleic acid.

In some examples, there is provided a method of amplification of a target nucleic acid, the method comprising annealing the target nucleic acid in the presence of a reverse primer of the target nucleic acid and a control nucleic acid, subjecting the target nucleic acid to reverse transcription, and subjecting the target nucleic acid to one or more amplification step in the presence of a mixture comprising a surfactant and a primer comprising one or more RNA base and a cleavable 3′ end.

### EXPERIMENTAL SECTION

In the present study, the inventors aim to demonstrate the feasibility of cfRNA in tracking thyroid tissue volume as a proof-of-concept to allow for further clinical study design to 1) examine the clinical utility in thyroid cancer risk stratification for guidance on RAI treatment, and 2) to assess its performance in detecting thyroid cancer recurrence.

The inventors of the present disclosure have identified 11 biologically significant and highly expressed thyroid-specific targets from the Human Protein Atlas and the current literature. To assess for a decrease in thyroid-specific cfRNA levels, the inventor of the present disclosure recruited 16 patients undergoing thyroid surgery, or RAI for malignant or benign thyroid disease, and tracked the longitudinal trends of cfRNA levels.

To assess the use of cfRNA levels in detecting metastatic thyroid cancer, cfRNA levels of 11 patients at intermediate to high risk of recurrence were measured during surveillance and clinical recurrence.

### MATERIAL & METHODS

### Clinical Sample Collection

In order to demonstrate a decrease in thyroid-specific cfRNA levels after thyroid surgery, 16 patients undergoing total or hemi-thyroidectomy for thyroid cancer or benign thyroid disease were recruited. Peripheral blood pre- and post-surgery was assessed for decrease in levels of circulating thyroid-specific cfRNAs. To establish the temporal trend of the cfRNA levels with thyroidectomy, cfRNA levels were evaluated at multiple time points of 24 hours, one week, one month, and six months post-surgery. There were two healthy volunteers included as the control group.

To assess the use of thyroid-specific cfRNA levels in detecting recurrent or persistent metastatic thyroid cancer, peripheral blood of 11 patients at American Thyroid Association (ATA) with intermediate to high recurrence of thyroid cancer were collected during surveillance visits and clinical recurrence. The cfRNA levels were correlated with serum TG levels, thyroid uptake on radioiodine scan (if available), and neck ultrasound.

Treatment response was classified as excellent, indeterminate, biochemical incomplete or structural incomplete based on the American Thyroid Association (ATA) thyroid cancer management guidelines. Patients were considered to have an excellent treatment response if they had non-stimulated TG<0.2 μg/L or stimulated serum TG<1.0 μg/L after RAI ablation, with no detectable TG Ab and no structural disease with the neck ultrasound scan and/or cross-sectional or radioiodine imaging. Patients were considered to have an indeterminate response if they had non-stimulated serum TG between 0.2 and 1.0 μg/L, stimulated serum TG≥1-10 μg/L after RAI ablation, stable or declining TG Ab or non-specific changes on neck ultrasound scan and/or cross-sectional or radioiodine imaging. Patients were considered to have biochemical incomplete treatment response if they had non-stimulated TG>1 μg/L after RAI ablation, stimulated serum TG>10 μg/L or increasing TG/TG Ab values without structural evidence of disease on imaging. Patients with structural evidence of disease on imaging were considered to have structural incomplete response.

The study was conducted from May 2018 to October 2020, and the study protocol was approved by the local ethics board committee (NHG DSRB Study Reference Number: 2017/00632). Written informed consent was obtained from all participants.

### Clinical Laboratory Measurement

Before 2015, serum TG levels were previously measured using the immunometric assay, e411 (Roche) or E170 (Roche), with a functional sensitivity of 0.5 μg/L. In 2015, the TG assay was changed to Kryptor (Brahms) with a functional sensitivity of 0.15 μg/L, and in 2017 to e411 (Roche) with a functional sensitivity of 0.1 μg/L. Before 2017, Anti-TG Abs were measured using radioimmunoassay Immulite 2000 (Siemens) assay, or from 2017 with e411 (Roche).

The analytical functional sensitivity of TG Ab was 20 IU/mL for the Immulite 2000 (Siemens) assay and the Roche assay. The inventors of the present disclosure considered a detectable level of TG Ab according to the functional sensitivity (limit of quantification) as TG Ab positive. Of note, reference values of TG Ab are reported to distinguish individuals with and without thyroid autoimmune disease, with detectable concentrations of TG Ab (i.e. above the functional sensitivity) even below the normal reference range to be considered as interfering with serum TG measurements.

### Selection of Thyroid-Specific Targets for Measurements in Plasma

A data-driven approach leveraging on thyroid-specific targets that is derived from the Human Protein Atlas was adopted to measure tissue-specific RNAs within the plasma. Genes that fall into the category of “Tissue-enriched genes” in thyroid tissues were selected. Genes that are highly expressed, with more than 4 times fold-change when compared to other tissues were selected. Selected targets were also verified in literature to be biologically relevant/significant before being included into the panel. Eventually, the following 11 thyroid-specific genes were targeted: TPO, TG, GFRA2, IYD, PDE8B, WDR86, C16orf89, DGKI, DIO2, TSHR, and PAX8.

### Circulating RNA Extraction

The use of consistent and reliable blood collection protocols is critical to maintain the integrity of circulating nucleic acid assays. The inventors of the present disclosure collected blood in Streck cfRNA tubes containing a stabilizing reagent to prevent cell lysis and to reduce RNA degradation.

cfRNA was extracted from 1 mL of plasma using a Plasma/Serum Circulating and Exosomal RNA Purification Kit (Norgen, Cat no. 42800). The residual DNA in the cfRNA was digested using an RNase-Free DNase I Kit (Norgen, Cat no. 25720). Extracted cfRNA was purified using an RNA Clean & Concentrator™-5 (Zymo, Cat no. ZYR.R1016), yielding 24 μL of cfRNA per sample.

### Reverse Transcription & Emulsion Based Targeted Pre-Amplification (FIG. 1)

10 μL of extracted cfRNA was annealed with 0.4 UM of reverse primers mix in the presence of 10,000 copies of Luciferase control RNA (Promega, Cat no. L4561) and 2 mM of dNTPs at 65° C. for 5 minutes. Reverse transcription of cfRNA was performed using Superscript™ III Reverse Transcriptase (Invitrogen, Cat no. 18080044) at 25° C. for 5 minutes, 50° C. for 50 minutes, and enzyme inactivation at 95° C. for 3 minutes.

cDNA from the reverse transcription was added to the PCR mixture of Platinum™ Taq DNA Polymerase (Invitrogen, Cat no. 10966) with 0.5 UM of rhPCR primers mix and 26 mU of RNase H2 enzyme. Emulsion was generated by adding 3 parts of 10% 008-FluoroSurfactant (RAN Biotechnologies) in 3M Fluorinert™ Engineered Fluid (3M, Cat no. FC-40) to 1 part of PCR reaction mixture. The mixture was vortexed until cloudy and uniform. Thermocycling includes enzyme activation at 94° C. for 2 minutes, followed by 20 cycles of denaturation (94° C., 15 seconds), annealing (55° C., 30 seconds), and extension (68° C., 1 minute). Reaction recovered from the emulsion PCR was topped up with the same amount of polymerase and RNase H2 used in the emulsion PCR. Second thermocycling reaction started with enzyme activation at 94° C. for 2 minutes, followed by 20 cycles of denaturation (94° C., 15 seconds), hybridization (78° C., 10 minutes), annealing (55° C., 30 seconds), and extension (68° C., 1 minute).

### Emulsion Breaking

Reaction was frozen at −80° C. for 1 hour before thawing at room temperature. The top fraction containing the reaction mix was transferred to a fresh tube. Fraction that was recovered was topped up with the same amount of Platinum™ Taq polymerase and RNase H2 enzyme used in the previous PCR reaction. Emulsion breaking may or may not be included in the Emulsion PCR step.

Quantification of Pre-Amplified Gene Targets Using qPCR

To monitor targeted cfRNA expression across different time points, qPCR was performed for 60 cycles with the Maxima SYBR Green/ROX qPCR Master Mix (ThermoFisher Scientific, Cat no. K0221).

### Spike-In Controls and Quality Control Assays for Ensuring Consistent Comparisons Across Patient Samples

There are two types of quality control. One for extraction efficiency and another for technical amplification efficiency. Extraction efficiency was normalized across samples using the levels of housekeeping gene such as, ACTB, GAPDH, and RPS18. Technical amplification efficiency was normalized across samples using a constant amount of spiked in Luciferase RNA that is not normally found in normal plasma (exogenous RNA). 10⁴ copies of Luciferase Control RNA (Promega, Cat no. L4561) was spiked in with 10 μL of the extracted cfRNA at the reverse transcription step to normalize for unintended variations in the experiment.

### Data Analysis

Data was analysed using normalized Ct values with respect to the housekeeping genes and are corrected for amplification efficiency using the amounts of spiked in luciferase RNA. All sequencing analysis was performed and plotted in R using ggplot. The inventors of the present disclosure normalized the raw Ct values with Luciferase control RNA and housekeeping genes, and verified the amplified product by referring to the melt curve. For an undetermined Ct/incorrect product, Ct value of 60 was assigned due to the 60 cycles of qPCR. The final Ct value was then deducted from 60 to allow easy visualization of the changes in RNA expression.

Levels of circulating cfRNA were represented as median with interquartile range. Statistical significance was determined according to p-values generated by Wilcoxon Signed Rank t test. Stata software (Version15.1; StataCorp, Texas, USA) was used for statistical analysis. The cfRNA level graphs were plotted using GraphPad Prism v9 (GraphPad Software).

### Results

### Validation of the Cot-Emulsion PCR Molecular Technology Protocol Using Artificial Spike in Controls

Luciferase RNA (LUC), which is not found in normal plasma RNA, is used as a spiked-in control to mimic the presence of low level of circulating RNA of interest across different applications. A range of LUC copies are used: 2¹⁶ [65536 molecules], 2¹⁸ [262144 molecules], 2²² [4,194,304 molecules] are spiked into RNA extracted from 1 ml of human plasma. These range of spiked-ins are used to illustrate the range of operability as well as scalability of the protocol.

These spiked in samples are put through 2 different versions of the molecular protocol. The first protocol comprises all the major steps including CoT amplification, to validate that the molecular technology is detecting the spike in LUC molecules. The second protocol has the CoT amplification process removed, to validate and illustrate the impact of CoT amplification in improving the Ct measurements and sensitivity of detection. In addition to LUC, housekeeping genes [RPS18, ACTB] are also used as positive controls for the platform.

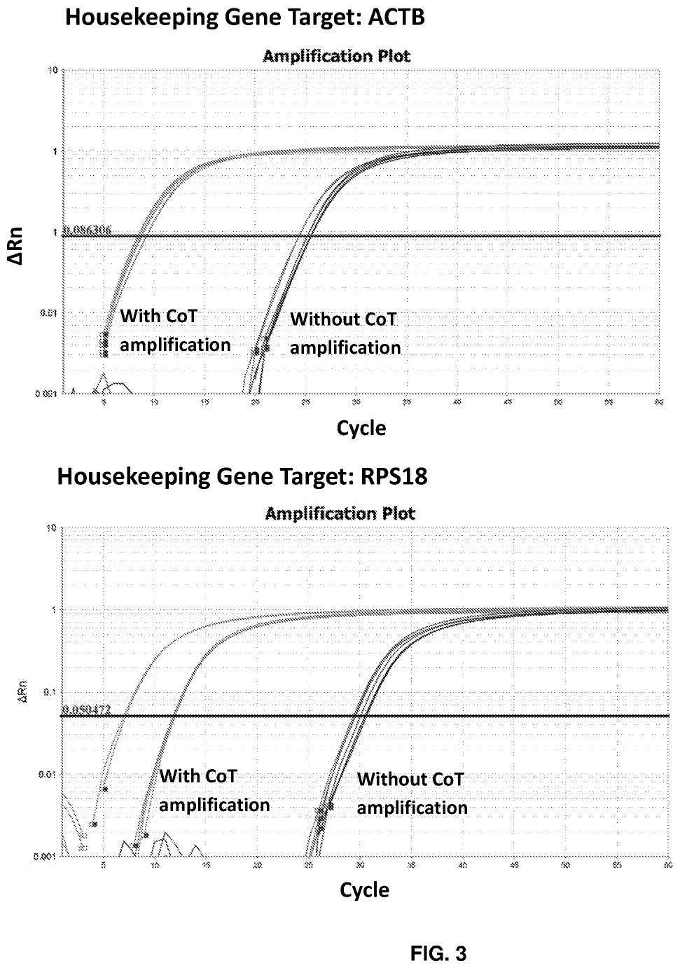

The inventors of the present disclosure show that the current protocol amplifies the target spiked in LUC and the quantified Ct cycles scales with the input range of molecules. In addition, CoT amplification significantly improves the sensitivity of the protocol by decreasing the Ct cycles. (FIG. 3 to FIG. 6).

### Multiplex Assay is Capable of Amplifying and Quantifying Multiple Thyroid-Specific Genes in a Single Reaction

RNA extracted directly from the thyroid was used as a positive control. Successful amplification of the targets collaborates the notion that the selected genes were highly expressed within the thyroid tissue (FIG. 7).

Thyroid-Specific RNA Transcripts are Present in the Plasma and in Amounts Quantifiable with qRT-PCR

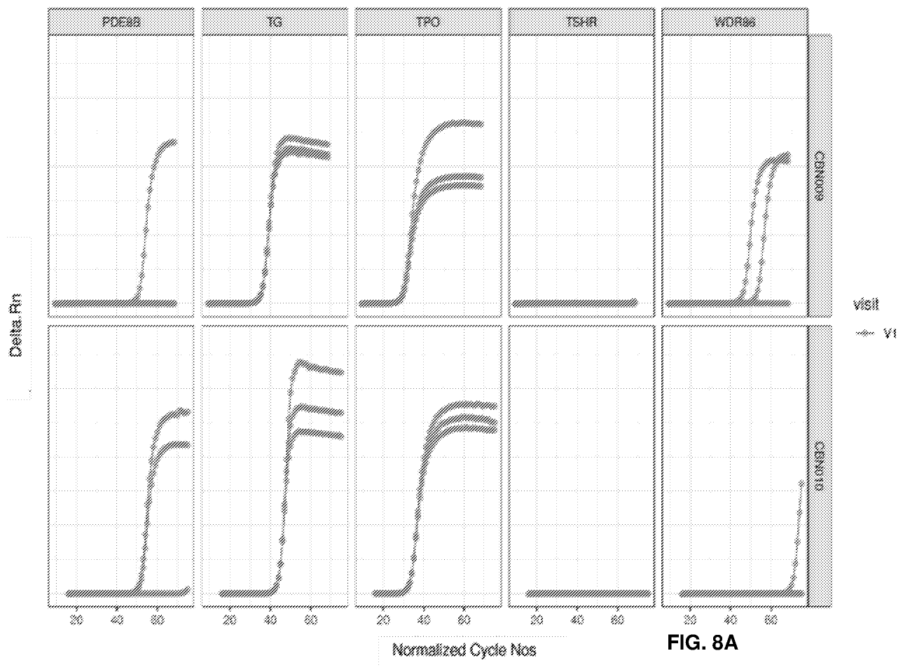

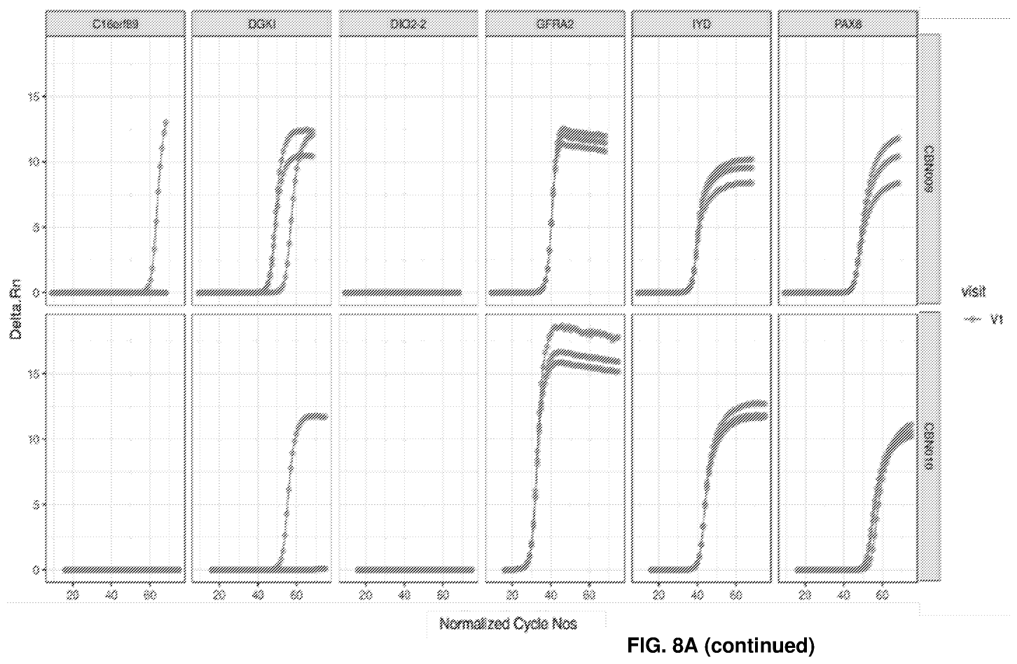

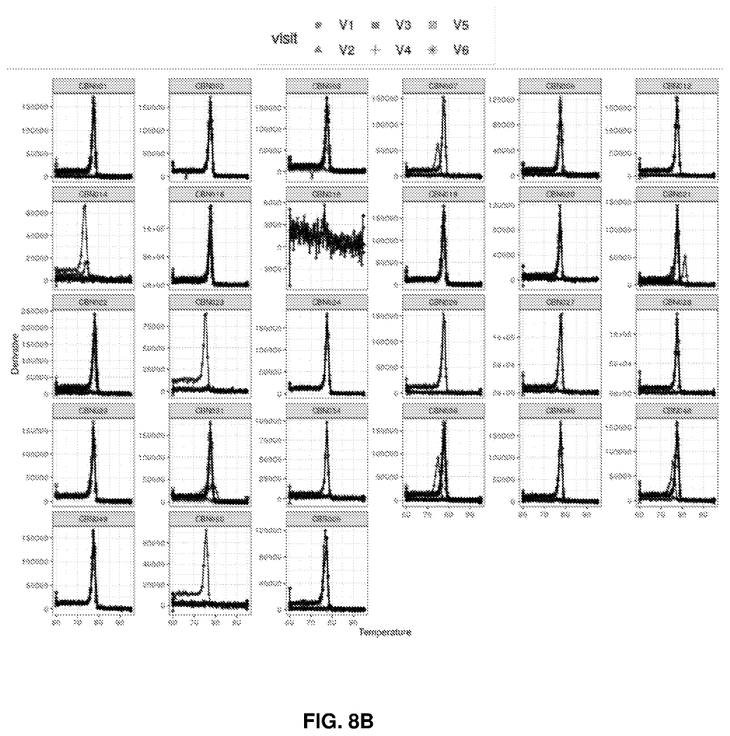

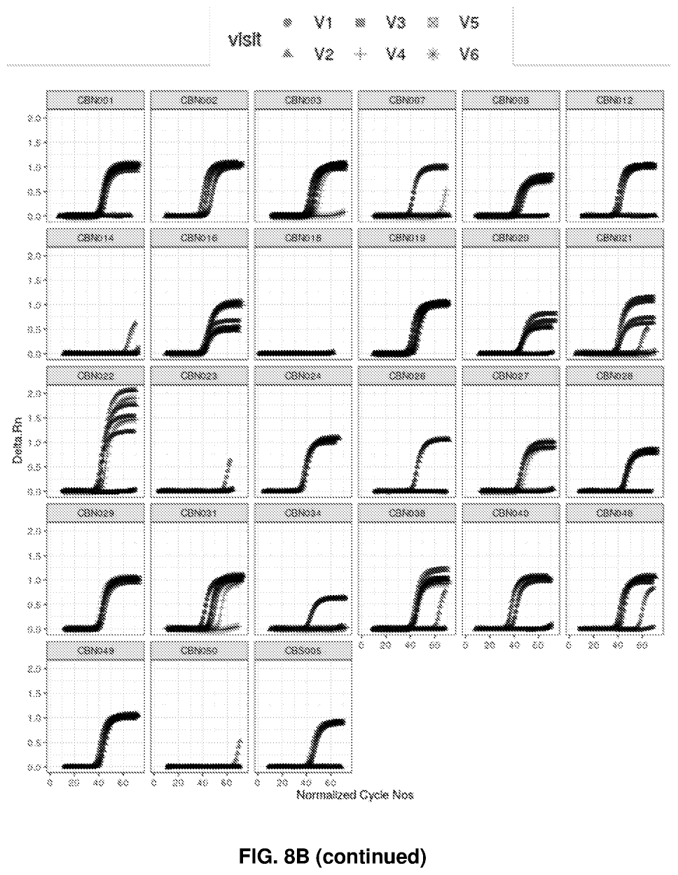

The 11 selected thyroid-specific RNA transcripts (TPO, TG, GFRA2, IYD, PDE8B, WDR86, C16orf89, DGKI, DIO2, TSHR, and PAX8) were amplified from healthy volunteers (FIG. 8A) and thyroid patients (FIG. 8B). Both circulating DIO2-2 RNA and TSHR RNA were not detectable in healthy controls (FIG. 8A). Similarly, in thyroid patients, only low levels of circulating DIO2-2 RNA were detected in two patients, with Ct values of 56 and 58 (data not shown). The circulating TSHR RNA was only detected in a single time point of 7 patients ( 7/16, 44%) with Ct values ranging from 36 to 58 (data not shown).

Thyroid-Specific cfRNA Levels Decrease Following Surgical or Pharmacological Ablation of the Thyroid Tissue

The inventors of the present disclosure evaluated the 11-circulating thyroid-specific cfRNA levels in patients undergoing thyroid surgery for i) benign thyroid conditions (e.g., benign thyroid nodules, hyperthyroidism from Graves' disease; n=3), ii) or malignant thyroid nodules (n=14), iii) and recurrent/persistent thyroid cancer undergoing repeat thyroid surgery or radioactive iodine adjuvant therapy (n=10). All the thyroid cancer patients had papillary thyroid cancer (PTC) except for a patient with both PTC and poorly differentiated thyroid cancer, and another patient with follicular thyroid cancer.

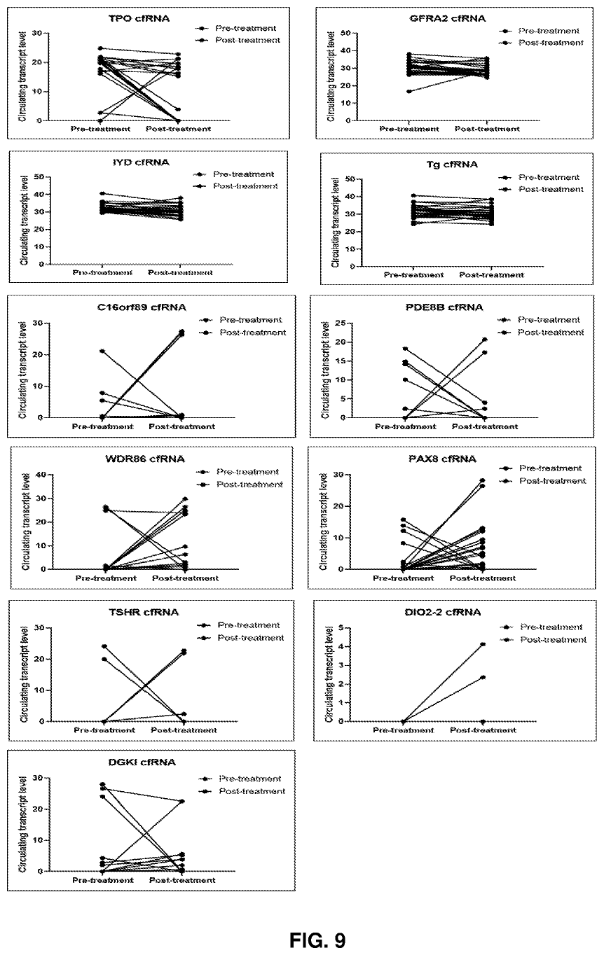

Four of the circulating plasma cfRNA transcript levels, namely TPO (thyroid peroxidase), IYD (iodotyrosine deiodinase), GFRA2 (glial cell line-derived neurotrophic factor family receptor alpha-2), and TG cfRNA, had decreased levels post-treatment in more than 50% of the study cohort, reaching statistical significance (Table 2; FIG. 9). The TPO cfRNA levels decreased post-therapy in 63% of the study cohort with a magnitude of decrease of 80%. Of note, in some patients, TPO cfRNA levels started to decrease as early as 1-day post-treatment. The magnitude of decrease in cfRNA levels post-treatment in IYD, TG, and GFRA2 cfRNA were less at 5% to 8%. Similar to TPO cfRNA levels, in some patients, IYD, TG, and GFRA2 cfRNA levels started to decrease as early as 1-day post-treatment, supporting the potential for cfRNAs to be early indicators of remnant thyroid tissue volume post-treatment.

TABLE 2
Circulating levels of thyroid-specific cfRNA transcript candidates pre- & post-treatment
in patients undergoing thyroidectomy or adjuvant radioactive iodine therapy. First
four rows represent cfRNA transcript levels that decrease post-treatment.

| | | | | | % | |
|---|---|---|---|---|---|---|
| | | | | | decrease | |
| | | | | median | in median | |
| | | | median | level | cfRNA | |
| | decrease | proportion | level pre- | post- | levels | |
| | with | of cohort | treatment | treatment | between | |
| | treatment | with | (interquartile | (interquartile | pre- & | |
| | (surgery | decrease | range) | range) | post- | |
| Total | or RAI) | post- | [60-Ct | [60-Ct | treatment | |
| n = 27 | (n) | treatment | value] | value] | levels | p value |
| TPO | 17 | 63% | 19.8 | 3.9 | −80% | 0.0079 |
| RNA | | | (2.7-21.3) | (0-18.6) | | |
| TG | 20 | 74% | 32.1 | 29.6 | −8% | 0.0264 |
| RNA | | | (29.4-33.6) | (28.3-33.6) | | |
| IYD | 20 | 74% | 31.8 | 30.3 | −5% | 0.0058 |
| RNA | | | (30.7-32.9) | (28.3-32.9) | | |
| GFRA2 | 19 | 70% | 30.4 | 28.2 | −7% | 0.0110 |
| RNA | | | (28.1-32.4) | (26.5-29.1) | | |
| WDR86 | 4 | 15% | 0 | 0 | — | |
| RNA | | | (0-0) | (0-6.3) | | |
| PAX8 | 5 | 19% | 0 | 1.9 | — | |
| RNA | | | (0-0.9) | (0-8.6) | | |
| DGKI | 4 | 15% | 0 | 0 | — | |
| RNA | | | (0-0) | (0-2.0) | | |
| PDE8B | 5 | 19% | 0 | 0 | — | |
| RNA | | | (0-0) | (0-0) | | |
| C16orf89 | 4 | 15% | 0 | 0 | — | |
| RNA | | | (0-0) | (0-0) | | |
| TSHR | 2 | 7% | 0 | 0 | — | |
| RNA | | | (0-0) | (0-0) | | |
| DIO2-2 | 0 | 0% | 0 | 0 | — | |
| RNA | | | (0-0) | (0-0) | | |

Thyroid-Specific cfRNA Transcripts Capture Temporal Trends in Clinical Course of Thyroid Cancer Patients

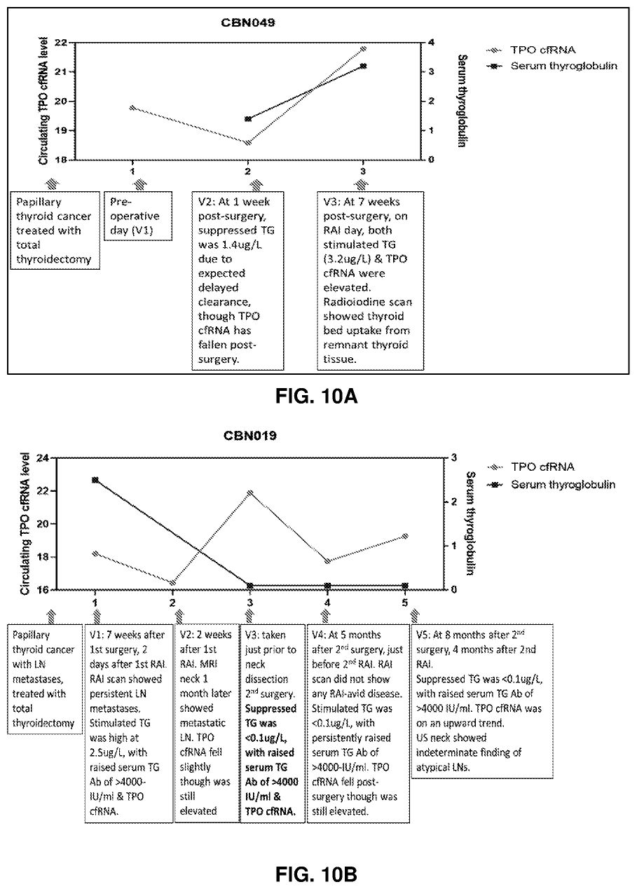

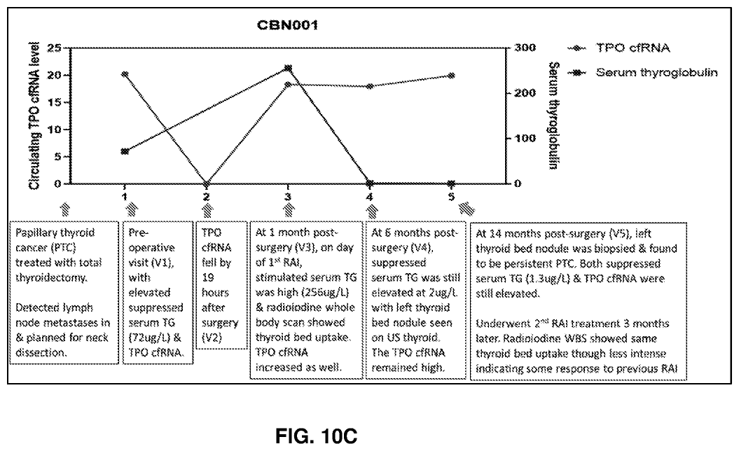

Temporal trends were showcased in 3 different clinical scenarios (peri-operative period, TG Ab positive state, recurrent/persistent thyroid cancer), reflecting different aspects where cfRNA measurements makes an impact. FIG. 10A, FIG. 10B and FIG. 10C show graphs of longitudinal trends of thyroid-specific cfRNA levels in the clinical course of thyroid cancer patients. The circulating cfRNA levels plotted along the Y-axis were derived from 60-Ct values.

The inventors of the present disclosure demonstrated the expected variability of the observed TPO cfRNA levels peri-operatively and under thyroid stimulating hormone (TSH) stimulation for adjuvant RAI therapy in a patient (CBN049) with Stage I papillary thyroid carcinoma [T3N0M0] (FIG. 10A). Time point 1 was assessed pre-operatively, and time point 2 was measured 1 week post-operatively showing a decrease in TPO cfRNA levels. The corresponding serum thyroglobulin level was 1.4 μg/L (not undetectable yet) as expected due to its delayed clearance. This patient did not have detectable TG Ab. As part of the standard of care, in order for maximal RAI influx into remnant thyroid tissues for ablation, the TSH level is intentionally kept elevated either through thyroid hormone withdrawal or through the administration of recombinant human TSH. Under these conditions, thyroid tissues are also stimulated to produce maximal amount of thyroid proteins, including thyroglobulin and TPO. This explains the elevated serum thyroglobulin and TPO cfRNA levels recorded at time point 3. This case illustrates that TPO cfRNA levels decreases after thyroid surgery, and TPO transcription is increased by TSH stimulation as indicated by increased circulating cfRNA levels.

Next, the inventors of the present disclosure reviewed the utility of cfRNA level measurements in a thyroid cancer patient with innate production of thyroglobulin antibodies (TG Ab) due to underlying autoimmune lymphocytic thyroiditis. In this clinical setting, TG Ab usually interferes with the serum thyroglobulin immunometric assay measurement. This patient (CBN019) had Stage I papillary thyroid carcinoma with cervical lymph node metastases [T2N1bM0] and was treated with total thyroidectomy and adjuvant RAI 7 weeks post-surgery (FIG. 10B). At time point 1 which was evaluated 2 days after RAI under the TSH stimulated state, serum TG levels was elevated at 2.5 μg/L, TG Ab levels was high at >4000 IU/ml (normal reference range is <115 IU/ml), and TPO cfRNA levels was also elevated. Theradioiodine scan showed persistent cervical lymph node metastases. At time point 2 which was measured 2 weeks after the first RAI, the patient's TPO cfRNA levels slightly decreased even though it was still elevated. MRI neck imaging performed 1 month after the patient's adjuvant RAI therapy confirmed persistent cervical lymph node metastases. Time point 3 was taken prior to the patient's neck dissection surgery for excision of the metastatic neck lymph nodes. The patient's TPO cfRNA levels at time point 3 had increased even though her serum TG was <0.1 μg/L. This is likely due to interference from the elevated TG Ab of >4000 IU/ml. At 5 months after the patient's second surgery (time point 4) which is the day of the second RAI in the TSH stimulated state, the patient's TPO cfRNA levels decreased even though it still remained elevated. The patient's stimulated TG levels was <0.1 μg/L with persistently elevated serum TG Ab of >4000-IU/ml. The radioiodine scan did not show any RAI-avid disease. At time point 5, which is 4 months after the second surgery, the patient's TPO cfRNA levels was on an upward trend with similar undetectable serum TG and elevated serum TG Ab of >4000-IU/ml. The neck ultrasound showed indeterminate finding of atypical lymph nodes that needed to be followed-up. This case illustrates the application of the cfRNA levels measurement technique for the detection of persistent thyroid cancer in the setting of positive serum TG Ab levels that interferes with TG immunometric assay.

The inventors of the present disclosure next analyzed cfRNA time course for a patient (CBN001) who had persistent papillary thyroid cancer despite initial total thyroidectomy (FIG. 10C). The patient had elevated serum TG levels post-surgery, in the absence of TG Ab, and was found to have cervical lymph node metastases. Prior to the neck dissection surgery, at time point 1, the patient had elevated serum TG (72 μg/L) and elevated TPO cfRNA levels. After the neck dissection surgery, TPO cfRNA levels decreased within a day (time point 2). On the day of adjuvant RAI (time point 3), in the TSH stimulated state, both the serum TG levels (256 μg/L) and TPO cfRNA levels were elevated. Theradioiodine scan showed thyroid bed RAI uptake indicating residual thyroid tissue. At time point 4, during surveillance at 6 months after neck dissection, both serum TG levels (2 μg/L) & TPO cfRNA levels were elevated, even though these levels were lower than the levels detected at time point 1 (before neck dissection and RAI). Ultrasound scan of the neck showed left thyroid bed nodule. This was later biopsied and proven to be persistent papillary thyroid cancer (time point 5). The biopsy needle washing TG levels was elevated at 5180 g/L, without detectable TG Ab. Concurrently, both serum TG levels (1.3 μg/L) and TPO cfRNA levels were elevated. Subsequently, the patient underwent another course of RAI treatment. The radioiodine scan showed a less intense thyroid bed RAI uptake, indicating that the previous RAI had partially ablated the tumour. This case illustrates the use of TPO cfRNA levels as a potential tool to detect recurrent or persistent thyroid cancer. The levels of TPO cfRNA could decrease within a day after treatment, indicating that measurements are instant snapshots of the molecular physiology in time.

### Key Study Findings

In this study, the inventors of the present disclosure aimed to assess the ability of circulating thyroid-specific cfRNAs in quantifying remnant thyroid gland tissue or malignancy. Circulating tissue specific RNAs had previously been reported to be present at low levels. To increase sensitivity of the assay, the inventors of the present disclosure measured multiple thyroid-specific targets using a multiplex pre-amplification approach. In addition, a data-driven bioinformatics approach was adopted to design a multiplex primer design assay specific to thyroid targets with minimal cross-interaction. Integrating multiple measurements targeting the thyroid, the inventors of the present disclosure found that TPO cfRNA is a potential circulating biomarker that could track the residual thyroid mass in patients, with levels decreasing dynamically as early as 1 day after treatment. The inventors of the present disclosure have demonstrated the clinical relevance of circulating TPO cfRNA by tracking the changes in levels throughout the patient's clinical course in the setting of peri-treatment, recurrence, and TG Ab positive state.

The identification of a circulating cfRNA that changes in levels and reflects molecular physiology in real-time, allows further clinical study design to 1) examine the clinical utility in remnant thyroid mass quantification for precise guidance on RAI doses, and 2) to assess its performance in detecting thyroid cancer recurrence, especially in patients with TG Ab that interferes with existing serum TG immunometric assays.

### Harnessing Thyroid-Specific Targets Beyond Thyroglobulin

The current standard of care relies on quantification of circulating thyroid-specific protein TG to reflect thyroid tissue mass. However, the most widely used TG assay, which is the immunometric assay underestimates serum TG levels in the presence of TG Ab due to the formation of TG-TG Ab complex that leads to a reduction of free TG, which is measured by immunometric assay. Several studies had assessed the utility of TG measurement by LC-MS/MS and showed the feasibility in detecting circulating TG via mass spectrometry. However, this needs to be further optimized to improve the current 40-60% level of sensitivity as there were patients with structural disease and TG Ab that were measured as having negative TG on LC-MS/MS. TG measurements with the radioimmunoassay (RIA) is an alternative technique for TG assessment in the presence of TG Ab, even though it was reported to be associated with false-positive TG measurements in patients with TG Ab. The assessment of circulating TG mRNA levels was shown to correctly identify 93% ( 13/14) of patients with structural disease, negative serum TG, in the presence of elevated TG Ab. This remains to be further validated.

The inventors of the present disclosure found that other circulating thyroid-specific targets such as TPO, IYD, TG, and GFRA2 cfRNA levels decreased accordingly post-treatment for thyroid cancer and could be potential biomarkers to reflect thyroid mass. Thyroglobulin (TG), thyroid peroxidase (TPO), and iodotyrosine deiodinase (IYD) are involved in thyroid hormone biosynthesis. TPO oxidizes iodide ions to iodine atoms for addition onto tyrosine residues on thyroglobulin, forming mono-iodotyrosine (MIT) and di-iodotyrosine (DIT). Thyroid hormones and thyroxine (T4) formation involves oxidative coupling of two DIT, whereas triiodothyronine (T3) de novo formation involves coupling of MIT and DIT. Some of the free MIT and DIT released by thyroid cells are scavenged by iodotyrosine deiodinase (also known as iodotyrosine dehalogenase 1, DEHAL1) to recycle iodide, thereby preventing iodide from leaking out of thyroid cells. As such, circulating levels of TPO, IYD, and TG cfRNA levels whose function are specific to the thyroid gland could plausibly reflect thyroid mass. GFRA2 was found to be highly expressed in brain and thyroid tissue and was enriched in papillary thyroid cancer from the TCGA dataset. GFRA2 mediates activation of the RET tyrosine kinase receptor and is a candidate gene for RET-associated diseases. GFRA2 was shown to be present on immunohistochemistry stain in case series of papillary, follicular, and medullary thyroid cancer, as well as follicular adenoma.

The current tumour marker serum TG takes at least 4 weeks for complete clearance whereas levels of TPO cfRNA changes as early as 1 day after treatment. With further optimization, TPO cfRNA levels could potentially allow early quantification of residual thyroid mass, and precise timely planning of adjuvant RAI dosage after thyroid surgery.

The quantification provided in the form of 60-ct values for circulating cfRNA levels does not vary proportionally to the expected amount of thyroid mass. For example, the levels of TPO cfRNA in patients with previous hemi-thyroidectomy is not half of the level in patients with an entire thyroid gland in situ. Furthermore, the cfRNA RT-qPCR technique requires further optimization for better precision. In addition, some patients with thyroid cancer did not shed TPO cfRNAs into the circulation and had measurable circulating TG, GFRA2, IYD cfRNA levels instead. As different clonal thyroid cancer cells could preferentially produce different thyroid proteins due to genetic aberrations, the use of a multiplex cfRNA panels that detects several thyroid-specific targets may be advantageous.

### DETAILED DESCRIPTION OF FIGURES

Example embodiments of the disclosure will be better understood and readily apparent to one of ordinary skill in the art from the following discussions and if applicable, in conjunction with the figures. It should be appreciated that other modifications related to structural, electrical and optical changes may be made without deviating from the scope of the invention. Example embodiments are not necessarily mutually exclusive as some may be combined with one or more embodiments to form new exemplary embodiments. The example embodiments should not be construed as limiting the scope of the disclosure.

FIG. 1 shows a schematic diagram of the workflow of reverse transcription and amplification of cfRNA. cfRNA, spiked-in with luciferase RNA control, was reverse transcript with Superscript™ III Reverse Transcriptase (Invitrogen, Cat no. 18080044). The product was amplified with rhPCR primers and Platinum™ Taq DNA Polymerase (Invitrogen, Cat no. 10966) using emulsion and CoT PCR. The residual primers were removed with Exonuclease I (New England Biolabs, Cat no. M0293). Amplified products were used for qPCR quantification and sequencing.

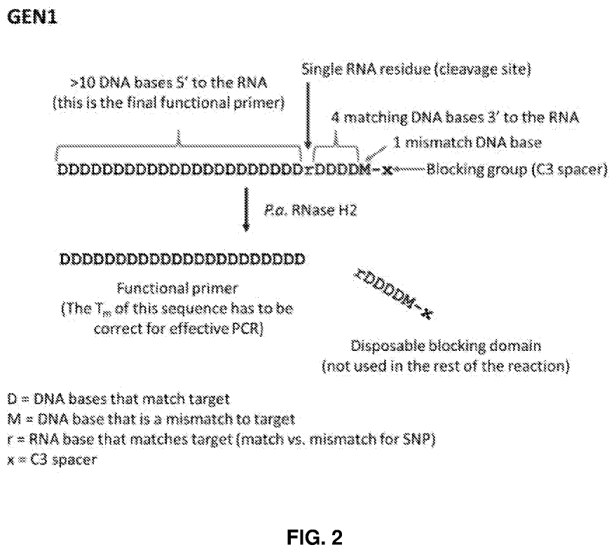

FIG. 2 shows the design of a rhPCR primer as used in the present disclosure. In one embodiment, the rhPCR primer may consists of five different parts: starting from the 5′ end with a functional primer, the cleavage site, four matching DNA bases, one mismatch DNA base, and a blocking group at the 3′ end. Cleavage of the RNA residue by RNase H2 will take place when the primer is perfectly complementary to the template, releasing the 3′ blocking group.

FIG. 3 shows Ct curves of housekeeping genes validating the protocol in amplification of housekeep genes found in plasma. Each curve is a biological replicate. Addition of CoT amplification decreases the CT cycle and improves sensitivity.

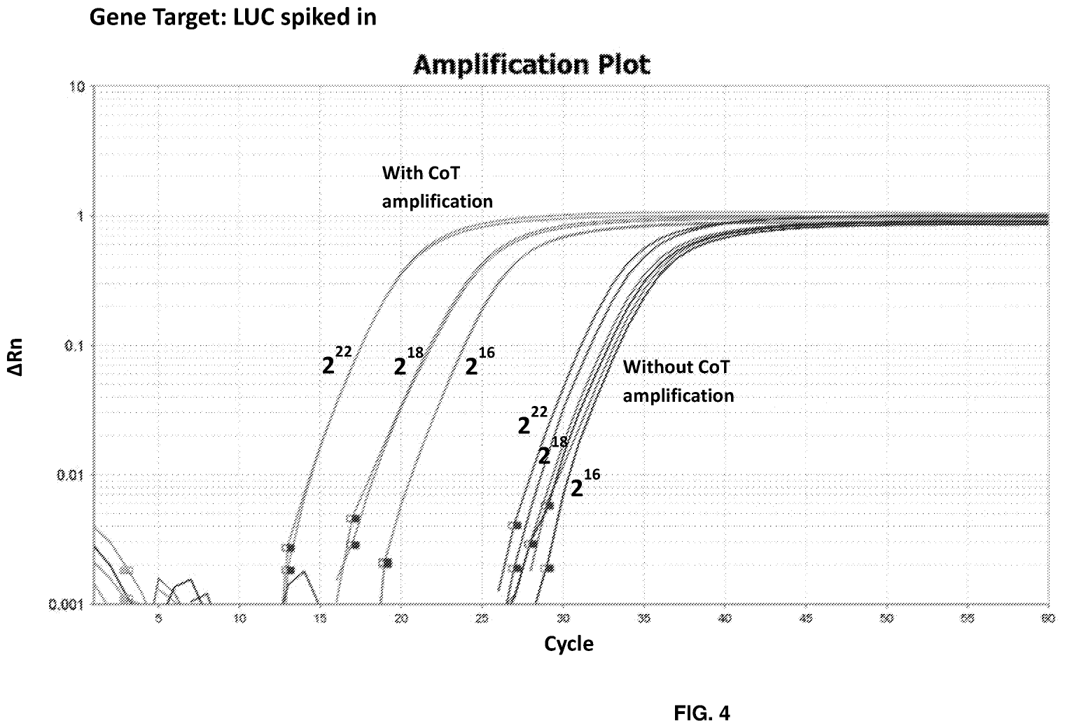

FIG. 4 shows Ct curves of LUC spiked-ins across a range of spiked in molecules. Each curve illustrates a different LUC spiked in. Addition of CoT amplification decreases the CT cycle and improves sensitivity.

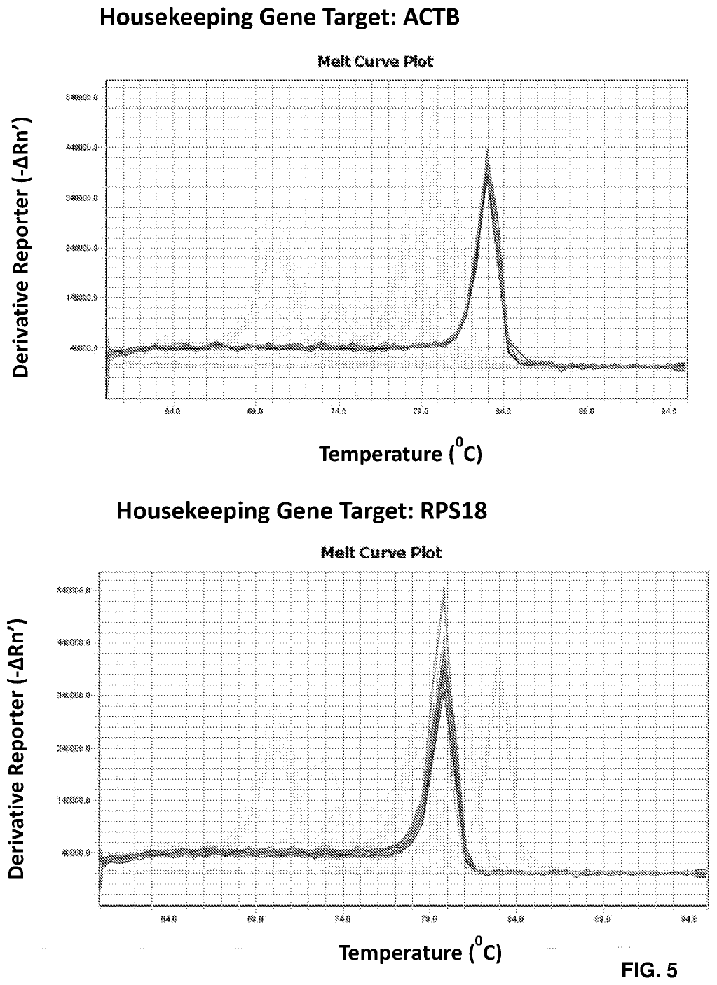

FIG. 5 shows melt curves of housekeeping genes validating the protocol in amplification of housekeep genes found in plasma. Each curve is a biological replicate. Sharp peaks indicating strong and specific amplification of the target at the intended melting temperature.

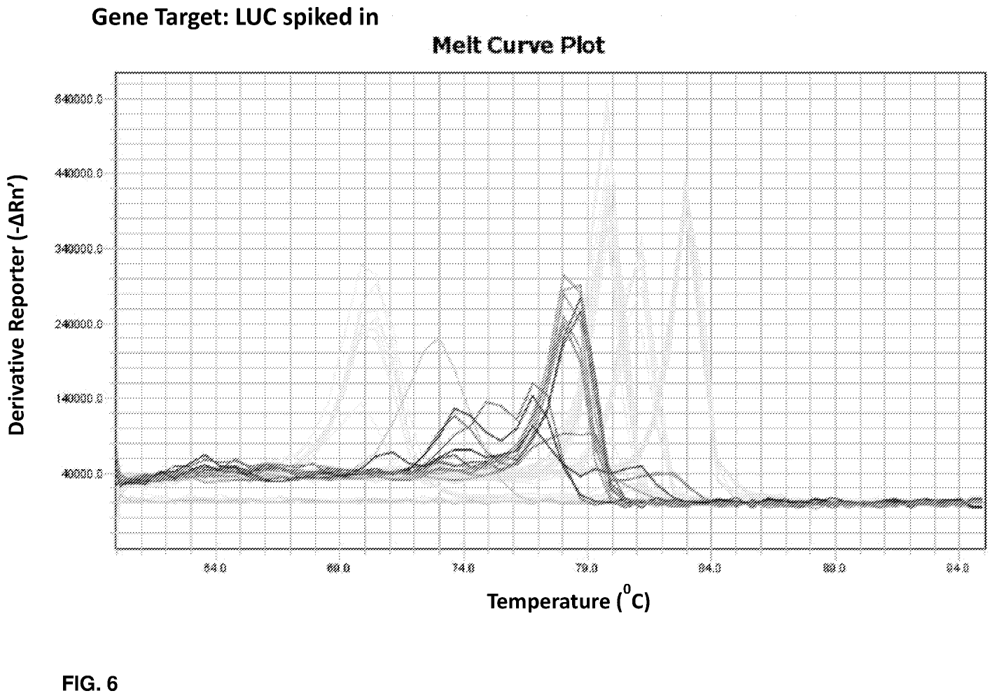

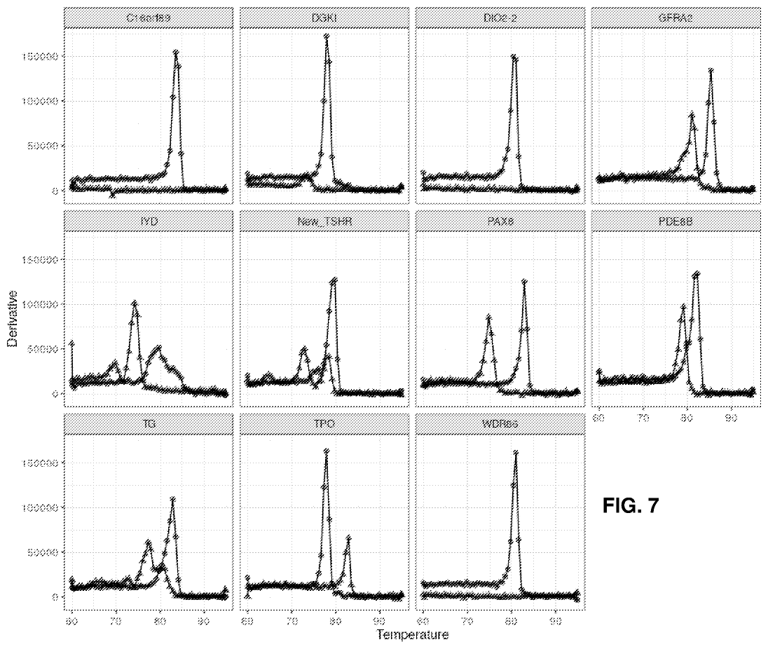

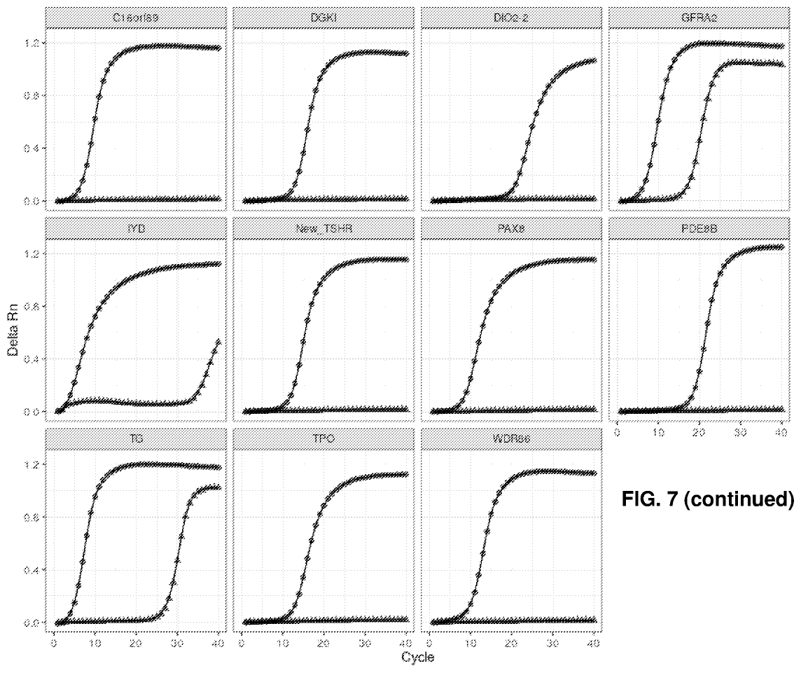

FIG. 6 shows melt curves of LUC spike-in targets validating the protocol in amplification of housekeep genes found in plasma. Each curve is a biological replicate. Sharp peaks indicating strong and specific amplification of the target at the intended melting temperature.

FIG. 7 shows the positive amplification curves and melt curves of all thyroid-specific RNA transcripts using directly extracted thyroid RNA.

FIG. 8A shows graphs of the Ct values in amplification curve of 11 thyroid-specific RNA transcripts across clinical plasma samples from 2 health volunteers.

FIG. 8B shows graphs of the Ct values of TPO across clinical plasma samples of thyroid patients, Amplification curve & melt curve of TPO.

FIG. 9 shows plots comparing pre- and post-treatment levels of thyroid-specific cfRNA transcripts candidates. The circulating cfRNA levels plotted along Y-axis were derived from 60-Ct values.

FIG. 10A shows a graph of peri-operative and pre-RAI TPO cfRNA levels and serum thyroglobulin levels at three time points in patients with papillary thyroid cancer.

FIG. 10B shows a graph of TPO cfRNA levels and serum thyroglobulin (TG) levels in the setting of positive thyroglobulin antibody (TG Ab) in patient with papillary thyroid cancer and lymph node (LN) metastases.

FIG. 10C shows a graph of TPO cfRNA levels and serum thyroglobulin (TG) levels in a patient with papillary thyroid cancer and persistent lymph node (LN) metastases undergoing further ablative therapy.

### APPLICATIONS

Embodiments of the methods disclosed herein provide a fast and efficient way of detecting a thyroid biomarker that can be found in a sample only in small amounts. Embodiments of the disclosed methods also seek to overcome the problems of providing a method of detecting thyroid biomarker with increased sensitivity.

Advantageously, the methods and/or mixtures as disclosed herein provides a one pot amplification of thyroid specific RNA from plasma where amplified cDNA is demonstrated to be compatible for downstream quantification using either qPCR or next generation sequencing.

Even more advantageously, the methods and/or mixtures as disclosed herein provides a robust non-chemical-based method of recovering emulsion PCR product using freeze-thaw cycle.

The present disclosure also advantageously provides for the inclusion of quality control method of normalizing PCR efficiency that utilizes spiked in luciferase RNA.

The present disclosure also provides an amplification (such as PCR) cycling protocol that leverages the CoT effect for increased sensitivity with minimal loss in linearity using in quantitation.

The present disclosure also provides an adaptable end point where amplified cDNA exhibits compatibility for downstream quantification using either qPCR or next-generation sequencing

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A method of detecting and/or determining the presence of one or more thyroid-specific nucleic acid, the method comprising:
annealing the one or more thyroid-specific nucleic acid in the presence of a control nucleic acid, and
subjecting each of the one or more thyroid-specific nucleic acid to one or more amplification step in the presence of a mixture comprising a surfactant and an oligonucleotide primer and/or probe capable of hybridizing with the one or more thyroid-specific nucleic acid, wherein the oligonucleotide primer and/or probe comprises a cleavage site and a cleavable 3′ end.

2. The method of claim 1, wherein the amplification step of the nucleic acid is performed in the presence of three parts surfactant to one part amplification mixture.

3. The method of claim 1, wherein method comprises two amplification steps.

4. The method of claim 1, wherein the method further comprises a step of freeze and thawing the amplified mixture.

5. The method of claim 1, wherein the method further comprises a step of freeze and thawing the amplified mixture between the one or more amplification steps.

6. The method of claim 1, wherein the control nucleic acid is added to the sample at a constant amount to thereby normalizes of the amplification efficiency across a plurality of samples, optionally the control nucleic acid is added to the sample at about 102 to 1010 copies.

7. The method of claim 1, wherein the amplification step includes interposing an annealing step between denaturation and priming.

8. The method of claim 1, wherein the method further comprises a step of quantifying the amount of one or more thyroid-specific nucleic acid present in the sample and/or sequencing the target nucleic acid in the sample.

9. The method of claim 1, wherein the one or more thyroid-specific nucleic acid is obtained from a biological sample.

10. The method of claim 1, wherein the one or more thyroid-specific nucleic acid is obtained from plasma.

11. The method of claim 1, wherein the one or more thyroid-specific nucleic acid is a cell free nucleic acid, optionally a circulating cell free nucleic acid, optionally a circulating cell free RNA.

12. The method of claim 1, wherein the thyroid-specific nucleic acid are genes that are highly expressed and/or have four or more folds-change expression in the thyroid as compared to in other tissues.

13. The method of claim 1, wherein the one or more thyroid-specific nucleic acid comprise thyroid peroxidase (TPO), thyroglobulin (TG), GFRA2 (glial cell line-derived neurotrophic factor family receptor alpha-2), IYD (iodotyrosine deiodinase), PDE8B (phosphodiesterase-8B), WDR86 (WD repeat domain 86), C16orf89 (Chromosome 16 Open Reading Frame 89), DGKI (Diacylglycerol kinase), DIO2 (Iodothyronine Deiodinase 2), TSHR (Thyroid Stimulating Hormone Receptor), and PAX8 (Paired box gene 8).

14. The method of claim 1, wherein the one or more thyroid-specific nucleic acid comprises thyroid peroxidase (TPO), GFRA2 (glial cell line-derived neurotrophic factor family receptor alpha-2), IYD (iodotyrosine deiodinase), and thyroglobulin (TG).

15. The method of claim 1, wherein the sample is obtained from a subject prior to and/or subsequent to a treatment, optionally the treatment is a surgery to remove thyroid and/or a radiation therapy.

16. The method of claim 1,
wherein the method comprises detecting and/or determining and/or quantifying the presence of the one or more thyroid-specific gene in a first sample and a second sample,
wherein the first sample is taken at an earlier time point than the second sample, and
wherein an increase in the presence of the one or more thyroid-specific gene in the second sample compared to the first sample indicates the subject to have residual thyroid tissue and/or tumour burden, or
wherein an increase in the presence of the one or more thyroid-specific gene in the second sample compared to the first sample indicates the subject to have recurrence and/or of thyroid conditions.

17. A method of detecting and/or determining thyroid cancer recurrence and/or metastasis, the method comprising:
annealing the one or more thyroid-specific nucleic acid in the presence of a control nucleic acid, and
subjecting each of the one or more thyroid-specific nucleic acid to one or more amplification step in the presence of a mixture comprising a surfactant and an oligonucleotide primer and/or probe capable of hybridizing with the one or more thyroid-specific nucleic acid, wherein the oligonucleotide primer and/or probe comprises a cleavage site and a cleavable 3′ end,
wherein the method comprises detecting and/or determining and/or quantifying the presence of the one or more thyroid-specific gene in a first sample and a second sample,
wherein the first sample is taken at an earlier time point than the second sample, and
wherein an increase in the presence of the one or more thyroid-specific gene in the second sample compared to the first sample indicates the subject to have thyroid cancer recurrence and/or metastasis.

18. The method of claim 17, wherein the one or more thyroid-specific gene comprises thyroid peroxidase (TPO), sodium-iodide symporter (NIS), thyroglobulin (TG), and thyroid stimulating hormone receptor (TSHR).

19. The method of claim 17, wherein the sample is a plasma sample.

20. A thyroid-specific nucleic acid detection mixture comprising
a first mixture comprising:
a control nucleic acid, and
a second mixture comprising:
a surfactant, and
an oligonucleotide primer and/or probe capable of hybridizing with a thyroid-specific nucleic acid, wherein the oligonucleotide primer and/or probe comprises a cleavage site and a cleavable 3′ end.
